(19) United States
(12) Patent Application Publication
(10) Pub. No.: US 2011/0027294 A1
(43) Pub. Date: Feb. 3, 2011

(54) Anti-Respiratory Syncytial Virus Antibodies, Antigens and Uses Thereof

(76) Inventors: Alfred Delvecchio, West Chester, PA (US); Ping Tsui, Berwyn, PA (US); Patrick Branigan, Lansdowne, PA (US); Leslee Conrad, Norristown, PA (US); Nicole Day, West Norriton, PA (US); Changbao Liu, Malvern, PA (US); Raymond Sweet, Bryn Mawr, PA (US); Sheng-Jiun Wu, Broomall, PA (US); Jose Melero, Madrid (ES); Jinquan Luo, Malvern, PA (US); Gabriela Canziani, Blue Bell, PA (US); Mark Tornetta, Collegeville, PA (US); Gopalan Raghunathan, San Diego, CA (US); Venkata Chalapathi Koka, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/900,924

(22) Filed: Oct. 8, 2010

Related U.S. Application Data
(62) Division of application No. 11/261,356, filed on Oct. 28, 2005.
(60) Provisional application No. 60/622,981, filed on Oct. 28, 2004.

(51) Int. Cl. A61K 39/155; C07K 16/10; A61K 39/42; C07H 21/04; C07K 7/08; A61P 31/14; C12N 15/63; C12P 21/02; C12N 5/10; C12N 5/20

(52) U.S. Cl. 424/159.1; 530/389.4; 530/387.9; 530/387.3; 530/391.1; 536/23.53; 530/326; 424/186.1; 435/320.1; 435/69.6; 435/339

(57) ABSTRACT

Anti-Respiratory Syncytial Virus (RSV) monoclonal antibodies, RSV F protein antigens and their uses are disclosed.

### CROSS REFERENCE TO RELATED APPLICATIONS

This application is a divisional of Ser. No. 11/261,356, filed 28 Oct. 2005, currently pending, which claims the benefit of U.S. Provisional Application No. 60/622,981, filed 28 Oct. 2004. The entire contents of each of the aforementioned applications are incorporated herein by reference.

### FIELD OF THE INVENTION

This invention relates to neutralizing antibodies to Respiratory Syncytial Virus (RSV) F protein and F protein peptides and their use to treat and prevent RSV-induced diseases.

### BACKGROUND OF THE INVENTION

Infection by human respiratory syncytial virus (RSV) has been long recognized as the single most common cause of serious lower respiratory tract infections (LRTIs) in infants, young children, immunocompromised individuals and more recently, the elderly. This pathogen is directly responsible for over 126,000 hospitalizations and an estimated 500 deaths of infants and young children annually in the United States (Shay et al., JAMA 282:1440-1446, 1999; Shay et al., J. Infect. Dis. 183:16-22, 2001). Furthermore, RSV infection is associated with the development of childhood wheezing and the exacerbation of asthma. Currently, an additional 14,000 to 60,000 RSV-related hospitalizations occur each year in the U.S. within the growing elderly segment, with a mortality rate of approximately 11% (reviewed in Dowell et al., J. Infect. Dis. 174:456-462, 1996; Han et al., J. Infect. Dis. 179:25-30, 1999). RSV infection is the most common viral respiratory infection of hematopoietic stem cell and solid organ transplant recipients and is associated with a wide range of mortality in this population (reviewed in Ison and Hayden, Curr. Opin. Infect. Dis., 15:355-367, 2002).

Neutralizing antibodies to RSV are directed against the viral F and G proteins present on the surface of the virus, with F representing the major protective antigen. The F protein is highly conserved (89% amino acid identity) between the two subgroups (A and B) of RSV and, in contrast to the G protein, protective antibody responses against F protein are cross-reactive between subgroups. In general, the majority of neutralizing antibodies to RSV F protein map to two regions of the protein designated site II and site IV,V,VI (Arbiza et al., J. Gen. Virol., 73:2225-2234, 1992; Lopez et al., J. Virol. 72:6922-6928, 1993; Collins et al., in Virology, vol. 1, 4^{th} ed., pp. 1443-1485, 2001).

A protective RSV vaccine has not yet been developed. Moreover, no effective therapeutic antiviral agents currently exist.

Synagis® brand of palivizumab, a RSV-neutralizing, humanized monoclonal antibody (mAb) targeting the viral F protein is marketed by MedImmune Inc. for the passive immunoprophylaxis of at risk infants (<35 weeks term, those with bronchopulmonary dysplasia (BPD) or congenital heart disease). See Johnson, S. et al., J. Infect. Dis., 176:1215-1224, 1997 and U.S. Pat. No. 5,824,307. However, immunoprophylaxis with Synagis® only reduces hospitalization rates in these at-risk infants overall by 55% (Impact-RSV Study Group, Pediatrics, 102:531-537, 1998) and significantly less well in those with BPD (39% reduction). As such, there is a significant unmet medical need for agents that effectively prevent and treat RSV infections.

### BRIEF DESCRIPTION OF THE DRAWINGS

FIGS. 1a (SEQ ID NO:3)_and 1b (SEQ ID NO:5)_show the nucleic acid sequence of the anti-RSV mAb 101F heavy and light chains, respectively. The CDRs are underlined in each chain.

FIGS. 2a (SEQ ID NO:4)_and 2b (SEQ ID NO:6)_show the predicted amino acid sequence of the anti-RSV mAb 101F heavy and light chains, respectively. FIGS. 2c (SEQ ID NO:49)_and 2d (SEQ ID NO:51)_show the predicted amino acid sequence of the anti-RSV mAb B21M heavy and light chains, respectively. The CDRs are underlined.

FIG. 3 shows binding of the anti-RSV mAb 101F to RSV F protein.

FIG. 4 shows binding of a chimeric anti-RSV mAb 101F and chimeric 101F Fab to RSV F protein.

FIG. 5 shows RSV neutralization activity of anti-RSV mAb 101F in a cotton rat immunoprophylaxis model.

FIG. 6 shows ELISA binding of anti-RSV F mAbs to synthetic peptides (SEQ ID NOs: 29-37 and 39-45). Panels A and B show 101F mAb binding; Panel C shows palivizumab binding to peptides 2555 to 2563.

7 shows relative binding affinity of 101F mAb to synthetic peptides (SEQ ID NOs: 29-37 and 39-45).

FIG. 8 shows ELISA binding to human RSV F protein of light chain variant Fabs relative to wild-type B23 Fab (E. coli optimized B21M Fab).

FIG. 9 shows Fab inhibition of RSV fusion by light chain variant Fabs relative to wild-type B23 Fab.

FIG. 10 shows antiviral activity of polyethylene glycol conjugated anti-RSV mAb.

### SUMMARY OF THE INVENTION

One aspect of the invention is an isolated antibody reactive with Respiratory Syncitial Virus (RSV) F protein having the antigen binding ability of a monoclonal antibody having the amino acid sequences of the heavy chain complementarity determining regions (CDRs) as set forth in SEQ ID NOs: 8, 10 and 12 and the amino acid sequences of the light chain CDRs as set forth in SEQ ID NOs: 14, 16 and 18.

Another aspect of the invention is an isolated antibody reactive with RSV isolates that escape neutralization by antibodies or antibody fragments containing CDRs derived from palivizumab or mAb19.

Another aspect of the invention is an isolated antibody reactive with an RSV F protein epitope located at residues 422 to 436 (SEQ ID NO: 28) of the F protein.

Another aspect of the invention is an isolated antibody reactive with residues R429 and K433 of an RSV F protein.

Another aspect of the invention is an isolated antibody having Hc-CDR1, Hc-CDR2 and Hc-CDR3 amino acid sequences as shown in SEQ ID NOs: 8, 10 and 12, respectively and a Lc-CDR1 as shown in Formula (I):

(I)
Arg Ala Ser Xaa₀ Ser Val Asp Xaa₁ Xaa₂ Gly Xaa₃
Ser Tyr Xaa₄ His

wherein Xaa₀ is Gln, Asp or His; Xaa₁ is Leu, His, Val, Phe or Tyr; Xaa₂ is Phe, Leu or Ser; Xaa₃ is Arg, Lys, Gln, Val, Gly, Thr or Ser; and Xaa₄ is Val or Met; and light chain CDR2 (Lc-CDR2) and light chain CDR3 (Lc-CDR3) amino acid sequences as shown in SEQ ID NOs: 16 and 18, respectively.

Another aspect of the invention is an isolated antibody having a Hc-CDR1 amino acid sequence as shown in SEQ ID NO: 8, a Hc-CDR2 amino acid sequence as shown in Formula (II):

(II)
His Xaa₇ Tyr Trp Asp Asp Asp Xaa₈ Arg Tyr Xaa₉ Pro
Ser Leu Lys Ser

wherein Xaa₇ is Ile or Leu, Xaa₈ is Lys or Tyr and Xaa₉ is Asn or Ser, a Hc-CDR3 amino acid sequences as shown in SEQ ID NO: 12 and a Lc-CDR1 and Lc-CDR2 amino acid sequence as shown in SEQ ID NOs: 14 and 16, respectively and a Lc-CDR3 amino acid sequence as shown in SEQ ID NO: 107 or 110.

Another aspect of the invention is an isolated antibody having Hc-CDR1 and Hc-CDR2 amino acid sequences as shown in SEQ ID NOs: 8 and 10, respectively and a Hc-CDR3 amino acid sequence as shown in Formula (III):

| | |
|---|---|
| Leu Xaa₅ Gly Phe Xaa₆ Tyr Gly Phe Ala Tyr | (III) |

wherein Xaa₅ is Tyr or Trp; and Xaa₆ is Arg, Lys or Ala and Lc-CDR1, Lc-CDR2 and Lc-CDR3 amino acid sequences are as shown in SEQ ID NOs: 14, 16 and 18, respectively.

Another aspect of the invention is an isolated antibody having

Hc-CDR1 and Hc-CDR2 amino acid sequences as shown in SEQ ID NOs: 8 and 10, respectively and a Hc-CDR3 amino acid sequence as shown in Formula (IV), a Lc-CDR1 amino acid sequence as shown in Formula (I) and Lc-CDR2 and Lc-CDR3 amino acid sequences as shown in SEQ ID NOs: 16 and 18, respectively.

Another aspect of the invention is an isolated antibody having a Hc-CDR1, Hc-CDR2 and Hc-CDR3 amino acid sequence as shown in SEQ ID NOs: 8, 10 and 12, respectively and a Lc-CDR1 amino acid sequence as shown in Formula (I) wherein Xaa₀ is Gln or Asp, Xaa₁ is Leu or Tyr, Xaa₂ is Phe, Xaa₃ is Lys or Arg and Xaa₄ is Met, a Lc-CDR2 amino acid sequence as shown in SEQ ID NO: 16 and a Lc-CDR3 amino acid sequence as shown in SEQ ID NO: 107.

Another aspect of the invention is an isolated antibody having a Hc-CDR1 amino acid sequence as shown in SEQ ID NO: 8, a Hc-CDR2 of formula II, a Hc-CDR3 of formula III, a Lc-CDR1 of formula I, a Lc-CDR2 amino acid sequence as shown in SEQ ID NO: 16 and a Lc-CDR3 amino acid sequence as shown in SEQ ID NO: 107.

Another aspect of the invention is an isolated antibody having a Hc-CDR1 amino acid sequence as shown in SEQ ID NO: 8, a Hc-CDR2 of formula II, a Hc-CDR3 of formula III, a Lc-CDR1 of formula I, a Lc-CDR2 amino acid sequence as shown in SEQ ID NO: 16 and a Lc-CDR3 amino acid sequence as shown in SEQ ID NO: 18.

Another aspect of the invention is an isolated polynucleotide encoding an antibody of the invention.

Another aspect of the invention is a method of preventing RSV-induced disease comprising administering, to a patient at risk thereof, a prophylactically effective amount of an antibody of the invention.

Another aspect of the invention is a method of treating RSV-induced disease comprising administering to a patient a therapeutically effective amount of an antibody of the invention.

Another aspect of the invention is an isolated polypeptide comprising the peptide CTASNKNRGIIKTFS (SEQ ID NO: 38).

Another aspect of the invention is an isolated nucleic acid encoding the amino acid sequence of SEQ ID NO: 38.

### DETAILED DESCRIPTION OF THE INVENTION

All publications, including but not limited to patents and patent applications, cited in this specification are herein incorporated by reference as though fully set forth.

The term “antibodies” as used herein is meant in a broad sense and includes immunoglobulin or antibody molecules including polyclonal antibodies, monoclonal antibodies including murine, human, humanized and chimeric monoclonal antibodies and antibody fragments.

In general, antibodies are proteins or polypeptides that exhibit binding specificity to a specific antigen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄.

The term “antibody fragments” means a portion of an intact antibody, generally the antigen binding or variable region of the intact antibody. Examples of antibody fragments include Fab, Fab′, F(ab′)₂ and Fv fragments, diabodies, single chain antibody molecules, such as scFv molecules where the variable heavy and variable light chains are connected as a single polypeptide chain by a linker and multispecific antibodies formed from at least two intact antibodies.

The term “antigen” as used herein means any molecule that has the ability to generate antibodies either directly or indirectly. Included within the definition of “antigen” is a protein-encoding nucleic acid.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

CDRs provide the majority of contact residues for the binding of the antibody to the antigen or epitope. CDRs of interest in this invention are derived from donor antibody variable heavy and light chain sequences, and include analogs and variants of the naturally occurring CDRs. When present in an antibody, analog CDRs retain the same antigen binding specificity and/or neutralizing ability as the donor antibody from which they were derived. Variant CDRs, when present in an antibody, confer improved antigen binding and/or neutralizing ability relevant to the donor antibody from which they were derived.

The term “in combination with” as used herein and in the claims means that the described agents can be administered to a mammal together in a mixture, concurrently as single agents or sequentially as single agents in any order.

The term “monoclonal antibody” (mAb) as used herein means an antibody (or antibody fragment) obtained from a population of substantially homogeneous antibodies. Monoclonal antibodies are highly specific, typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256:495-497 (1975). Chimeric mAbs containing a light chain and heavy chain variable region derived from a donor antibody (typically murine) in association with light and heavy chain constant regions derived from an acceptor antibody (typically another mammalian species such as human) can be prepared by the method disclosed in U.S. Pat. No. 4,816,567. Humanized mAbs having CDRs derived from a non-human donor immunoglobulin (typically murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl Acad Sci (USA), 86:10029-10032 (1989) and Hodgson et al., Bio/Technology, 9:421 (1991). Exemplary human framework sequences useful for humanization are disclosed at, e.g., www.ncbi.nlm.nih.gov/entrez/query.fcgi; www.ncbi.nih.gov/igblast; www.atcc.org/phage/hdb.html; www.mrc-cpe.cam.ac.uk/ALIGNMENTS.php; www.kabatdatabase.com/top.html; ftp.ncbi.nih.gov/repository/kabat; www.sciquest.com; www.abcam.com; www.antibodyresource.com/onlinecomp.html; www.public.iastate.edu/˜pedro/research tools.html; www.whfreeman.com/immunology/CH05/kuby05.htm; www.hhmi.org/grants/lectures/1996/vlab; www.path.cam.ac.uk/˜mrc7/mikeimages.html; mcb.harvard.edu/BioLinks/Immunology.html; www.immunologylink.com; pathbox.wustl.edu/˜hcenter/index.html; www.appliedbiosystems.com; www.nal.usda.gov/awic/pubs/antibody; www.m.ehime-u.ac.jp/˜yasuhito/Elisa.html; www.biodesign.com; www.cancerresearchuk.org; www.biotech.ufl.edu; www.isac-net.org; baserv.uci.kun.nl/˜jraats/links1.html; www.recab.uni-hd.de/immuno.bme.nwu.edu; www.mrc-cpe.cam.ac.uk; www.ibt.unam.mx/vir/V_mice.html; http://www.bioinf.org.uk/abs; antibody.bath.ac.uk; www.unizh.ch; www.cryst.bbk.ac.uk/˜ubcg07s; www.nimr.mrc.ac.uk/CC/ccaewg/ccaewg.html; www.path.cam.ac.uk/˜mrc7/humanisation/TAHHP.html; www.ibt.unam.mx/vir/structure/stat_aim.html; www.biosci.missouri.edu/smithgp/index.html; www.jerini.de; imgt.cines.fr; Kabat et al., Sequences of Proteins of Immunological Interest, U.S. Dept. Health (1983), each entirely incorporated herein by reference.

The term “RSV neutralizing activity” as used herein refers to an antibody or antibody fragment that inhibits the ability of RSV to infect cells or to spread from an infected cell to an uninfected cell.

Conventional one and three-letter amino acid codes are used herein as follows:

| Amino acid | Three-letter code | One-letter code |
|---|---|---|
| Alanine | ala | A |
| Arginine | arg | R |
| Asparagine | asn | N |
| Aspartate | asp | D |
| Cysteine | cys | C |
| Glutamate | glu | E |
| Glutamine | gln | Q |
| Glycine | gly | G |
| Histidine | his | H |
| Isoleucine | ile | I |
| Leucine | leu | L |
| Lysine | lys | K |
| Methionine | met | M |
| Phenylalanine | phe | F |
| Proline | pro | P |
| Serine | ser | S |
| Threonine | thr | T |
| Tryptophan | trp | W |
| Tyrosine | tyr | Y |
| Valine | val | V |

The present invention relates to antibodies with RSV neutralizing activity that bind the RSV F protein (SEQ ID NOs: 1 and 2). The F (fusion) antigen is expressed both on the surface of the RSV particle and on the surface of cells infected with RSV and mediates the fusion of infected cells into syncytia. The binding epitope of the antibodies of the invention is located in the F protein region ₄₂₀TKCTASNKNRGIIKTFSNGCDYVSNK₄₄₅ (SEQ ID NO: 28). More specifically, the antibodies of the invention bind residues ₄₂₂CTASNKNRGIIKTFS₄₃₆ (SEQ ID NO: 38) of the RSV F protein. In particular, the antibodies of the invention bind residues R429 and K433 of the RSV F protein. These antibodies are useful as potential therapeutic or prophylactic agents for the treatment or prevention of RSV infection in mammals such as humans. These antibodies are also useful as research or diagnostic reagents.

Another embodiment of the invention is an isolated polypeptide comprising a peptide having the amino acid sequence CTASNKNRGIIKTFS (SEQ ID NO: 38) and an isolated nucleic acid encoding the polypeptide or its complement. These polypeptides and nucleic acids of the invention are useful as antigens in vaccine preparations to elicit neutralizing antibodies against RSV in a subject thereby immunizing the patient against RSV-induced disease. Any such vaccine preparations would be formulated and contain appropriate adjuvant as is well known to those skilled in the art.

Another embodiment of the invention is an isolated antibody reactive with RSV F protein having the binding ability of a monoclonal antibody having heavy chain CDR1 (hc-CDR1), CDR2 (hc-CDR2) and CDR3 (hc-CDR3) amino acid sequences as shown in SEQ ID NOs: 8, 10 and 12, respectively and light chain CDR1 (lc-CDR1), CDR2 (lc-CDR2) and CDR3 (lc-CDR3) amino acid sequences as shown in SEQ ID NOs: 14, 16 and 18, respectively. An exemplary antibody is a monoclonal antibody having hc-CDR1, hc-CDR2 and hc-CDR3 amino acid sequences as shown in SEQ ID NOs: 8, 10 and 12, respectively and lc-CDR1, lc-CDR2 and lc-CDR3 CDR amino acid sequences as shown in SEQ ID NOs: 14, 16 and 18, respectively.

Another embodiment of the present invention is an isolated monoclonal antibody having a heavy chain variable region (V_{H}) amino acid sequence as shown in SEQ ID NO: 4 and a light chain variable region (V_{L}) amino acid sequence as shown in SEQ ID NO: 6. Another embodiment of the invention is a nucleic acid encoding the amino acid sequences shown in SEQ ID NO: 4 or SEQ ID NO: 6 or its complement. An exemplary nucleic acid sequence encoding the amino acid sequence shown in SEQ ID NO: 4 is shown in SEQ ID NO: 3. An exemplary nucleic acid sequence encoding the amino acid sequence shown in SEQ ID NO: 6 is shown in SEQ ID NO: 5.

Yet another embodiment of the invention is a isolated nucleic acid encoding an antibody heavy chain having the hc-CDR1, hc-CDR2 and hc-CDR3 amino acid sequences shown in SEQ ID NOs: 8, 10 and 12, respectively or a complementary nucleic acid. An exemplary nucleic acid sequence has the nucleic acid sequences shown in SEQ ID NOs: 7, 9 and 11 encoding the amino acid sequences shown in SEQ ID NOs: 8, 10 and 12, respectively.

Yet another embodiment of the invention is a isolated nucleic acid encoding an antibody light chain having the lc-CDR1, lc-CDR2 and lc-CDR3 amino acid sequences shown in SEQ ID NOs: 14, 16 and 18, respectively or a complementary nucleic acid. An exemplary nucleic acid sequence has the nucleic acid sequences shown in SEQ ID NOs: 13, 15 and 16 encoding the amino acid sequences shown in SEQ ID NOs: 14, 16 and 18, respectively.

Another embodiment of the present invention is a human-adapted monoclonal antibody having a V_{H} amino acid sequence as shown in SEQ ID NO: 49 and a V_{L} amino acid sequence as shown in SEQ ID NO: 51.

Another embodiment of the present invention is an antibody having Hc-CDR1, Hc-CDR2 and Hc-CDR3 amino acid sequences as shown in SEQ ID NOs: 8, 10 and 12, respectively and a Lc-CDR1 as shown in Formula (I):

(I)
Arg Ala Ser Xaa₀ Ser Val Asp Xaa₁ Xaa₂ Gly Xaa₃
Ser Tyr Xaa₄ His

wherein Xaa₀ is Gln, Asp or His; Xaa₁ is Leu, His, Val, Phe or Tyr; Xaa₂ is Phe, Leu or Ser; Xaa₂ is Arg, Lys, Gln, Val, Gly, Thr or Ser; and Xaa₄ is Val or Met; and Lc-CDR2 and Lc-CDR3 amino acid sequences as shown in SEQ ID NOs: 16 and 18, respectively. Exemplary species include antibodies having a V_{H} amino acid sequence as shown in SEQ ID NO: 49 and a V_{L} amino acid sequence comprising a Lc-CDR2 and Lc-CDR3 amino acid sequences as shown in SEQ ID NOs: 16 and 18, respectively and a Lc-CDR1 of Formula (I) where:

(SEQ ID NO: 63)
Xaa₀ is Gln, Xaa₁ is Leu, Xaa₂ is Phe, Xaa₃ is Arg and Xaa₄ is Met;
(SEQ ID NO: 64)
Xaa₀ is Gln, Xaa₁ is Leu, Xaa₂ is Phe, Xaa₃ is Lys and Xaa₄ is Met;
(SEQ ID NO: 65)
Xaa₀ is Gln, Xaa₁ is Leu, Xaa₂ is Phe, Xaa₃ is Gln and Xaa₄ is Met;
(SEQ ID NO: 66)
Xaa₀ is Gln, Xaa₁ is Leu, Xaa₂ is Phe, Xaa₃ is Val and Xaa₄ is Met;
(SEQ ID NO: 67)
Xaa₀ is Gln, Xaa₁ is His, Xaa₂ is Phe, Xaa₃ is Lys and Xaa⁴ is Val;
(SEQ ID NO: 68)
Xaa₀ is Gln, Xaa₁ is Val, Xaa₂ is Phe, Xaa₃ is Arg and Xaa₄ is Met;
(SEQ ID NO: 105)
Xaa₀ is Gln, Xaa₁ is Leu, Xaa₂ is Leu, Xaa₃ is Gly and Xaa₄ is Met;
(SEQ ID NO: 69)
Xaa₀ is Gln, Xaa₁ is Leu, Xaa₂ is Phe, Xaa₃ is Thr and Xaa₄ is Met;
(SEQ ID NO: 70)
Xaa₀ is Gln, Xaa₁ is Tyr, Xaa₂ is Leu, Xaa₃ is Arg and Xaa₄ is Met;
(SEQ ID NO: 71)
Xaa₀ is Gln, Xaa₁ is Leu, Xaa₂ is Phe, Xaa₃ is Ser and Xaa₄ is Met;
(SEQ ID NO: 72)
Xaa₀ is Gln, Xaa₁ is Tyr, Xaa₂ is Ser, Xaa₃ is Arg and Xaa₄ is Met;
(SEQ ID NO: 73)
Xaa₀ is Gln, Xaa₁ is Phe, Xaa₂ is Phe, Xaa₃ is Lys and Xaa₄ is Met;
and
(SEQ ID NO: 111)
Xaa₀ is Asp, Xaa₁ is Leu, Xaa₂ is Phe, Xaa₃ is Val and Xaa₄ is Met.

Another embodiment of the present invention is an isolated antibody having a Hc-CDR1 amino acid sequence as shown in SEQ ID NO: 8, a Hc-CDR2 amino acid sequence as shown in Formula (II):

(II)
His Xaa₇ Tyr Trp Asp Asp Asp Xaa₈ Arg Tyr Xaa₉ Pro
Ser Leu Lys Ser

where Xaa₇ is Ile or Leu, Xaa₈ is Lys or Tyr and Xaa₉ is Asn or Ser, a Hc-CDR3 amino acid sequences as shown in SEQ ID NO: 12 and a Lc-CDR1 and Lc-CDR2 amino acid sequence as shown in SEQ ID NOs: 14 and 16, respectively and a Lc-CDR3 amino acid sequence of Gln Gln Ile Ile Asp Asp Pro Trp Thr as shown in SEQ ID NO: 107 or Gln Gln Ile Ile Ala Asp Pro Trp Thr as shown in SEQ ID NO: 110. Exemplary species include antibodies having a V_{L} amino acid sequence as shown in SEQ ID NO: 74 and a V_{H} amino acid sequence as shown in SEQ ID NO: 106.

Another embodiment of the present invention is an isolated antibody having Hc-CDR1 and Hc-CDR2 amino acid sequences as shown in SEQ ID NOs: 8 and 10, respectively and a Hc-CDR3 amino acid sequence as shown in Formula (III):

| | |
|---|---|
| Leu Xaa₅ Gly Phe Xaa₆ Tyr Gly Phe Ala Tyr | (III) |

wherein Xaa₅ is Tyr or Trp; and Xaa₆ is Arg, Lys or Ala and Lc-CDR1, Lc-CDR2 and Lc-CDR3 amino acid sequences are as shown in SEQ ID NOs: 14, 16 and 18, respectively. Exemplary species include antibodies having a V_{L} amino acid sequence as shown in SEQ ID NO: 51 and a V_{H} amino acid sequence comprising a Hc-CDR3 of Formula (IV) where:

| | |
|---|---|
| Xaa₅ is Trp and Xaa₆ is Arg; | (SEQ ID NO: 75) |
| Xaa₅ is Trp and Xaa₆ is Lys; | (SEQ ID NO: 76) |
| Xaa₅ is Trp and Xaa₆ is Ala; | (SEQ ID NO: 77) |
| Xaa₅ is Tyr and Xaa₆ is Arg; | (SEQ ID NO: 78) |
| Xaa₅ is Tyr and Xaa₆ is Lys; | (SEQ ID NO: 79) |
| and | |
| Xaa₅ is Tyr and Xaa₆ is Ala. | (SEQ ID NO: 80) |

Another embodiment of the present invention is an isolated antibody having Hc-CDR1 and Hc-CDR2 amino acid sequences as shown in SEQ ID NOs: 8 and 10, respectively and a Hc-CDR3 amino acid sequence as shown in Formula (IV), a Lc-CDR1 amino acid sequence as shown in Formula (I) and Lc-CDR2 and Lc-CDR3 amino acid sequences as shown in SEQ ID NOs: 16 and 18, respectively. Exemplary species include antibodies having a V_{L} amino acid sequence as shown in SEQ ID NO: 63 and a V_{H} amino acid sequence as shown in SEQ ID NOs: 76, 80 or 79.

Another embodiment of the present invention is an antibody having a Hc-CDR1, Hc-CDR2 and Hc-CDR3 amino acid sequence as shown in SEQ ID NOs: 8, 10 and 12, respectively and a Lc-CDR1 amino acid sequence as shown in Formula (I) wherein Xaa₀ is Gln or Asp, Xaa₁ is Leu or Tyr, Xaa₂ is Phe, Xaa₃ is Lys or Arg and Xaa₄ is Met, a Lc-CDR2 amino acid sequence as shown in SEQ ID NO: 16 and a Lc-CDR3 amino acid sequence as shown in SEQ ID NO: 107. Exemplary species include antibodies having a V_{L} amino acid sequence as shown in SEQ ID NOs: 81, 82, 83 or 112 and a V_{H} amino acid sequence as shown in SEQ ID NO: 49.

Another embodiment of the present invention is an antibody having a Hc-CDR1 amino acid sequence as shown in SEQ ID NO: 8, a Hc-CDR2 of formula II, a Hc-CDR3 of formula III, a Lc-CDR1 of formula I, a Lc-CDR2 amino acid sequence as shown in SEQ ID NO: 16 and a Lc-CDR3 amino acid sequence as shown in SEQ ID NO: 107 or 110. Exemplary species include antibodies having one of the following V_{L} and V_{H} amino acid sequence combinations:

| V_{L} SEQ ID NO: | V_{H} SEQ ID NO: |
|---|---|
| 83 | 84 |
| 85 | 86 |
| 81 | 86 |
| 85 | 84 |
| 87 | 88 |
| 89 | 86 |
| 85 | 88 |
| 91 | 86 |
| 92 | 93 |
| 94 | 86 |
| 95 | 88 |
| 96 | 88 |
| 97 | 86 |
| 81 | 88 |
| 99 | 88 |
| 87 | 49 |
| 100 | 88 |
| 101 | 88 |
| 102 | 88 |
| 102 | 49 |
| 103 | 86 |
| 103 | 88 |
| 108 | 88 |
| 90 | 88 |
| 85 | 88 |
| 109 | 88 |
| 63 | 88 |
| 85 | 93 |
| 94 | 93 |
| 112 | 88 |

Another embodiment of the present invention is an antibody having a Hc-CDR1 amino acid sequence as shown in SEQ ID NO: 8, a Hc-CDR2 of formula II, a Hc-CDR3 of formula III, a Lc-CDR1 of formula I, a Lc-CDR2 amino acid sequence as shown in SEQ ID NO: 16 and a Lc-CDR3 amino acid sequence as shown in SEQ ID NO: 18. Exemplary species include antibodies having one of the following V_{L} and V_{H} amino acid sequence combinations:

| V_{L} SEQ ID NO: | V_{H} SEQ ID NO: |
|---|---|
| 90 | 86 |
| 90 | 88 |
| 98 | 49 |
| 63 | 77 |

The antibodies of the invention can be conjugated to polyethylene glycol (PEGylated) to improve their pharmacokinetic profiles. Conjugation can be carried out by techniques known to those skilled in the art. Conjugation of therapeutic antibodies with PEG has been shown to enhance pharmacodynamics while not interfering with function. See Deckert et al., Int. J. Cancer 87: 382-390, 2000; Knight et al., Platelets 15: 409-418, 2004; Leong et al., Cytokine 16: 106-119, 2001; and Yang et al., Protein Eng. 16: 761-770, 2003. Pharmacokinetic properties of the antibodies of the invention could also be enhanced through Fc modifications by techniques known to those skilled in the art.

Another embodiment of the invention is an isolated nucleic acid encoding the heavy and light chains of any one of the antibodies of the invention or its complement. Exemplary nucleic acids include those encoding the V_{L} regions having any one of the amino acid sequences of SEQ ID NOs: 51, 63, 64, 65, 66, 67, 68, 104, 69, 70, 71, 72, 73, 74, 81, 82, 83, 85, 87, 89, 90, 91, 92, 94, 95, 96, 97, 98, 99, 100, 101, 102 and 103. Other exemplary nucleic acids include encoding the V_{H} regions having any of the amino acid sequences of SEQ ID NOs: 49, 106, 75, 76, 77, 78, 79, 80, 84, 86, 88 and 93.

Exemplary plasmid vectors useful to produce the antibodies of the invention contain a strong promoter, such as the HCMV immediate early enhancer/promoter or the MHC class I promoter, an intron to enhance processing of the transcript, such as the HCMV immediate early gene intron A, and a polyadenylation (polyA) signal, such as the late SV40 polyA signal. The plasmid can be multicistronic to enable expression of both the full-length heavy and light chains of the antibody, a single chain Fv fragment or other immunoglobulin fragments.

The mode of administration for therapeutic or prophylactic use of the anti-RSV antibodies of the invention may be any suitable route which delivers the agent to the host. The antibodies, antibody fragments and pharmaceutical compositions of these agents can be delivered by parenteral administration, i.e., subcutaneously, intramuscularly, intradermally, intravenously or intranasally as well as by topical or aerosol routes for delivery directly to target organs such as the lungs.

Anti-RSV antibodies of the invention may be prepared as pharmaceutical compositions containing an effective amount of the agent as an active ingredient in a pharmaceutically acceptable carrier. An aqueous suspension or solution containing the agent, preferably buffered at physiological pH, in a form ready for injection is preferred. The compositions for parenteral administration will commonly comprise a solution of the binding agent of the invention or a cocktail thereof dissolved in a pharmaceutically acceptable carrier, preferably an aqueous carrier. A variety of aqueous carriers may be employed, e.g., 0.4% saline, 0.3% glycine and the like.

Solutions of these pharmaceutical compositions are sterile and generally free of particulate matter. These solutions may be sterilized by conventional sterilization techniques (e.g., filtration). The compositions may contain pharmaceutically acceptable auxiliary substances as required to approximate physiological conditions such as pH adjusting and buffering agents, etc. The concentration of the anti-RSV antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or, more particularly, about 5 mg to about 25 mg of an anti-RSV antibody of the invention. Similarly, a pharmaceutical composition of the invention for intravenous infusion could be made up to contain about 250 ml of sterile Ringer's solution, and about 1 mg to about 30 mg or, more particularly, about 5 mg to about 25 mg of an anti-RSV antibody of the invention. Actual methods for preparing parenterally administrable compositions are well known or will be apparent to those skilled in the art and are described in more detail in, e.g., “Remington: The Science and Practice of Pharmacy (Formerly Remington's Pharmaceutical Sciences)”, 19th ed., Mack Publishing Company, Easton, Pa. (1995).

The anti-RSV antibody of the invention, when in a pharmaceutical preparation, can be present in unit dose forms. The appropriate therapeutically effective dose can be determined readily by those of skill in the art. A determined dose may, if necessary, be repeated at appropriate time intervals selected as appropriate by a physician during the treatment period.

The anti-RSV antibody of the invention can be lyophilized for storage and reconstituted in a suitable carrier prior to use. This technique has been shown to be effective with conventional immunoglobulins and protein preparations and art-known lyophilization and reconstitution techniques can be employed.

The present invention will now be described with reference to the following specific, non-limiting examples.

### Example 1

### Generation and Selection of an Anti-RSV mAb 101F

Anti-RSV mAbs were generated in normal BALB/c mice using standard hybridoma technology (Kohler et al., Nature 256: 495-497 (1975)). Mice were immunized essentially as previously described (Garcia-Barreno et al., J. Virol. 63:925-932, 1989) using a combinantion of purified virus (Long strain) and purified F protein (derived from RSV Long strain).

Three days prior to B cell fusion, female BALB/c mice were given an intravenous injection of the immunogen in PBS). Spleens from immunized mice were harvested and B cell fusion with Sp2/0 myeloma cells was carried out using standard methods of Kohler et al., supra. Fused cells were selected using medium containing hypoxanthine-aminopterin-thymidine and wells were screened for the presence of anti-RSV F antibodies by enzyme-linked immunosorbent assay (ELISA) using either purified virus or purified F protein as the antigen. Positive wells are expanded and cloned by limiting dilution.

Anti-RSV mAbs were selected based on their ability to bind using either purified virus or purified F protein as the antigen.

### Example 2

### Cloning and Sequencing of 101F Heavy and Light Chain Variable Region Genes

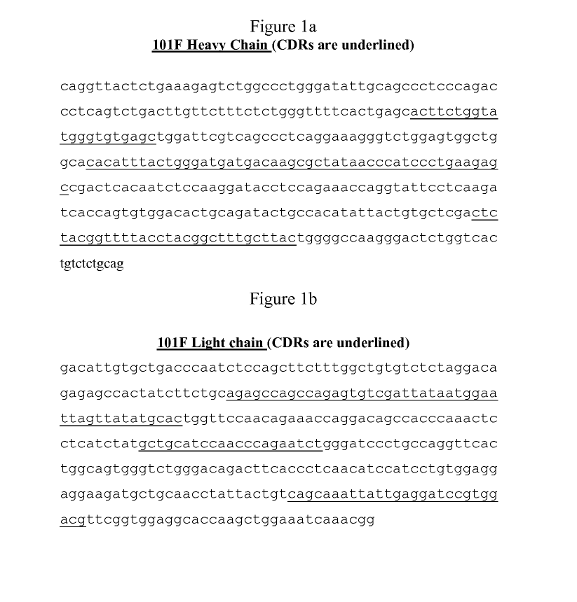

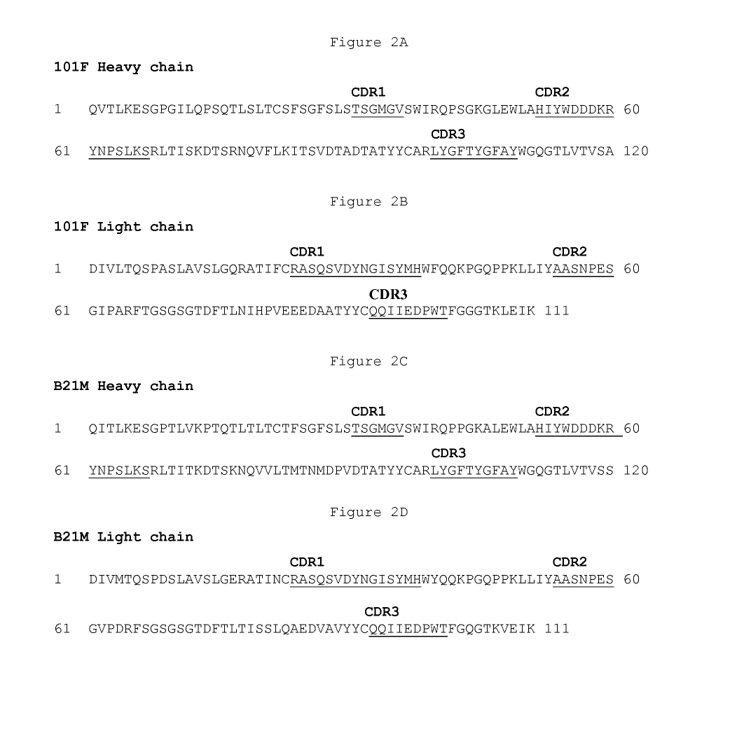

Total RNA from a hybridoma cell line expressing murine 101F IgG2a kappa was purified for cDNA generation using a GeneRacer Kit for 5′ RACE (InVitrogen, Carlsbad, Calif.). Oligo dT supplied with the kit primed the cDNA synthesis portion of the protocol. To obtain the heavy chain variable region gene, the cDNA was used as template in a touchdown PCR reaction according to the manufacturer's instructions. Equal amounts of GeneRacer 5′ primer (5′-CGACTGGAGCACGAGGACACTGA-3′) (SEQ ID NO: 19) and rat IgG2a 3′ primer 642 (5′-CTGTCCCGAGGTCTCAAGG-3′) (SEQ ID NO: 20) generated the expected size band of 660 by after the reaction. The same template and reaction conditions were used to obtain the light chain variable region. Equal amounts of the GeneRacer 5′ primer (SEQ ID NO: 19) and rat kappa 3′ primer 645 (5′-GAACTGTGACTACAGAGACC-3′) (SEQ ID NO: 21) generated the expected size band of 700 base pairs. Both heavy and light chain amplifications were performed in duplicate. Eight DNA preps of both heavy and light chain from individual colonies were sequenced using T3 and T7 primers. All eight heavy chain samples were confirmed to contain identical sequence. The majority of light chain samples (6 of 8) shared identical sequence, except for two that encoded a well-documented ‘pseudogene’ that resulted from the original fusion. FIGS. 1 and 2a and 2b show the nucleotide and deduced protein sequences respectively for the variable regions of the heavy (SEQ ID NOs: 3 and 4) and light chains (SEQ ID NOs: 5 and 6) of 101F.

### Example 3

### Cloning of Anti-RSV mAb 101F and Chimeric 101F Transient Constructs

The heavy chain variable region cDNA was used as a template in a polymerase chain reaction (PCR) using oligos 101HC5′ and 101HC3′ (sequences shown below).

101HC5′:
(SEQ ID NO: 22)
5′-TTCGTACGGCCACCATGGACAGGCTTACTTCCT-3′
101HC3′:
(SEQ ID NO: 23)
5′-TTCGAAACTTACCTGCAGAGACAGTGACCA-3′

These primers added a Kozak consensus sequence (underlined) to the translational start site (boldface) and appropriate restriction sites, BsiWI and BstBI (in italics). This amplified fragment was cloned into the BsiWI and BstBI sites of the murine genomic IgG2a expression plasmid p2370 containing the HCMV promoter and the SV40 polyadenylation site to generate the transient expression plasmid p2504 encoding the murine 101F IgG2a heavy chain. The light chain variable region cDNA was used as template and amplified with oligos LC101LIC5′ and LC101LIC3′ (sequences shown below), which add a Kozak consensus sequence (underlined) to the translational start site (boldface) and splice donor site (italics).

L101LIC5′:
(SEQ ID NO: 24)
5′GGTGCGTCCTTCGACCACCATGGAGTCAGACACACTCCTGC-3′
L101LIC3′:
(SEQ ID NO: 25)
5′-CGCCTCCGCTTCGACTTACGTTTGATTTCCAGCTTGGTGCC-3′

The resulting amplified 101F light chain variable region DNA fragment was cloned into a murine kappa chain expression vector by ligation independent cloning to generate p2505 encoding the murine 101F kappa light chain under the control of the HCMV promoter.

For the construction of the chimeric 101F (ch101F) mAb (murine variable regions grafted onto human IgG1 constant regions), the 101F heavy chain variable region was amplified by PCR using oligos H101LIC5′ and H101LIC3′ (sequences shown below) which add a Kozak consensus sequence (underlined) to the translational start site (boldface) and splice donor sequence (in italics) to the 5′ and 3′ ends of the DNA fragment respectively.

H101LIC5′:
(SEQ ID NO: 26)
5′-GGTGCGTCCTTCGACCACCATGGACAGGCTTACTTCCTCATT-3′
H101LIC3′:
(SEQ ID NO: 27)
5′-CGCCTCCGCTTCGACTTACCTGCAGAGACAGTGACCAGAGTCCC-3′

The heavy chain DNA fragment was cloned by ligation independent cloning into an expression vector generating plasmid p2535 encoding the murine 101F variable region grafted fused to a human IgG1 constant region under the control of the HCMV promoter. Plasmid p2536 which expresses the 101F light chain variable region fused to a human kappa light chain constant region was generated by cloning the same DNA fragment used to produce plasmid p2505 into a expression plasmid containing the human kappa light chain under the control of the HCMV promoter.

### Example 4

### Cloning of Anti-RSV mAb 101F and Chimeric Stable Constructs

The 101F heavy chain variable region was used to replace the variable region of plasmid p2521 that expresses the heavy chain of a murine anti-human CD4, IgG2a. The light chain variable region was cloned into plasmid p2527 that expresses the murine kappa light chain of an anti-human CD4, IgG2a. The resulting expression plasmids p2533 and p2534 express the heavy and light chains of murine 101F IgG2a respectively.

### Example 5

### Expression and Purification of Anti-RSV mAb 101F and Fabs

Plasmids designed for the stable expression of the 101F heavy and light chains were co-transfected by standard electroporation procedure into CD-Sp2/0 cells (C463A) and selected with mycophenolic acid. Supernatants from 96-well plates were assayed using standard EIA procedures using anti-murine or anti-human Fc-coated plates and alkaline phosphatase conjugated anti-murine or anti-human IgG (H+L) antibodies were used for detection. For transient expression of mAbs, 293E cells were transfected with Lipofectamine 2000 (Invitrogen, Inc., Carlsbad, Calif.) according to manufacturer's recommendations. To purify mAbs from hybridoma or transfected cell supernatants, 10× Dulbecco's phosphate-buffered saline (DPBS) without Mg⁺⁺ or Ca⁺⁺, pH 7.2 was added to clarified cell supernatants to a final concentration of 1× DPBS. The diluted supernatant was then loaded onto a 100 mL MabSelect™ column at 6.0 mls/min, which was equilibrated in 1× DPBS. The column was washed with 10 column volumes, or 1000 mls, of 1× DPBS. Antibodies were eluted using 0.1M glycine buffer, pH 2.5. Fractions as detected by increases in absorbance at 280 nm were collected into 2.5M Tris buffer, pH 7.5 to neutralize the eluted mAb. The purified mAb was dialyzed against 1× DPBS and sterilized by filtration through a 0.2 micron filter. The purity of the mAb preparations was analyzed by HPLC-SEC and electrophoresis through SDS-containing polyacrylamide gels (SDS-PAGE). The endotoxin levels of the preparations were determined using a Limulus amebocyte lysate (LAL) assay performed on the Pyros® Kinetix machine (Seikagaku America, East Falmouth, Mass.). Fabs from the various mAbs were generated by digestion at 37° C. for 4 hours with 2% papain and 2mM L-cysteine after which the reaction was stopped by the addition of 0.02M iodoacetamide. The Fabs were purified from the digestion mixture by protein-A affinity chromatography followed by size exclusion chromatography. The appropriate fractions containing the Fabs were pooled and analyzed by high-pressure liquid chromatography-size exclusion chromatography (HPLC-SEC) and SDS-PAGE.

### Example 6

### Anti-RSV mAb 101F Binding Activity

Binding of mAbs and Fabs to the soluble, extracellular domain of recombinantly expressed RSV F protein (Calder et al., Virology 271:122-131, 2000; Begona Ruiz-Anguello et al., Virology 298:317-328, 2002) was quantified by ELISA. Maxisorp plate wells (Nalge-Nunc, Rochester, N.Y.) were coated with fifty microliters (0.5 ug/ml) of purified RSV F protein in Tris-buffered saline (TBS, Teknova, Hollister, Calif.) by incubation at 4° C. overnight. The coated plate was then washed once with TBS-T (Tris Buffered Saline containing 0.05% Tween 20, Teknova #T0390) followed by the addition of a 1:1 mixture of ChemiBLOCKER™ (Chemicon, Temecula, Calif.) and SuperBlock® (Pierce, Rockford, Ill.) with shaking for one hour at room temperature. The plate was washed once with TBS-T followed by the addition of five-fold serial dilutions of antibodies in TBS containing 10 ug/ml of bovine serum albumin (BSA) up to a maximum concentration of 10 ug/ml. After one hour incubation at room temperature with shaking, plates were washed five times with TBS-T, followed by the addition of fifty microliters of a 1:4000 dilution of an alkaline phosphatase-conjugated goat anti-human IgG F(ab′)₂ (Jackson ImmunoResearch, West Grove, Pa.). After incubation at room temperature for one hour, fifty microliters of AttoPhos substrate (Roche Diagnostics, Indianapolis, Ind.) were added to the wells followed by incubation in the dark for ten minutes. Fluorescence was determined using a Tecan Spectra Fluor Plus (Tecan, Zurich Switzerland). To enhance detection sensitivity, the assay was performed as described above with the following modifications. A 1:4000 dilution of a biotin conjugated-goat anti-human IgG F(ab′)₂ (Jackson Immuno Research) was substituted for the alkaline phosphatase conjugated-goat anti-human IgG F(ab′)₂ described above followed by incubation at 22° C. for 1 hour with shaking. After five washes with TBS-T, the antibody binding was detected by the addition of fifty microliters of a 1:5000 dilution of alkaline phosphatase-conjugated streptavidin (ZyMed Laboratories, South San Francisco, Calif.) to each well for 45 minutes followed by five washes with TBS-T and detection as described above.

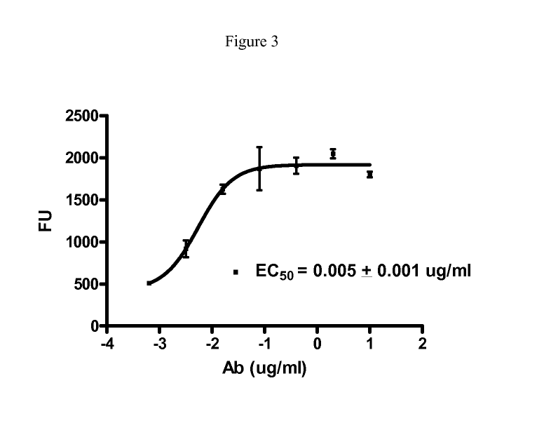

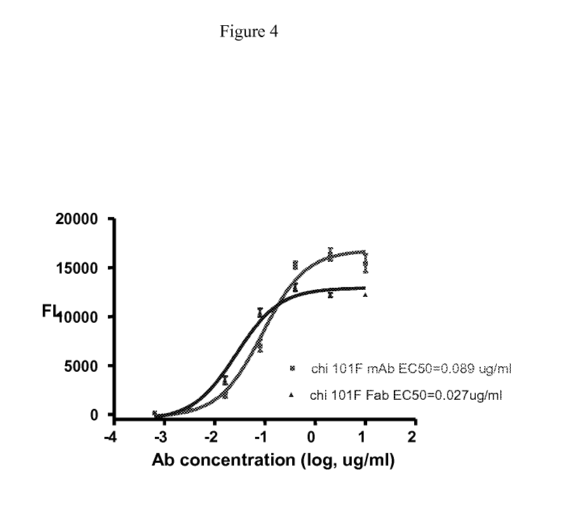

As shown in FIG. 3, the parental murine 101F mAb binds to recombinant RSV F protein with an EC50 of 5.5 ng/ml. FIG. 4 shows equivalent binding activity for the chimeric 101F and the chimeric 101F Fab to recombinant RSV F protein.

### Example 7

### Determination of Anti-RSV mAb 101F Affinity

A BIAcore 3000 instrument (Biacore Inc, Piscataway, N.J.) was used with a CM5 sensor chip. Running buffer contained 10 mM sodium phosphate 150 mM sodium chloride, pH 7.4, with 3 mM EDTA and 0.005% Tween-20 and all study experiments were performed at 25° C. Purified recombinant extracellular domain of RSV F-protein as previously described (Begona Ruiz-Anguello et al., supra) was used. F-protein was diluted into 300 microliters of 10 mM sodium acetate buffer, pH 4.5, to generate a 9.5 ug/mL final concentration for immobilization using NHS/EDC coupling reagents (BIAcore, Inc). The Application Wizard was programmed to be in the approximate range of 150 RU immobilized F-protein, with less than 200 RU scored as an acceptable parameter. After immobilization, the surface was washed using 50 mM sodium hydroxide. A surface modified with 1 M ethanolamine, pH 8.0 was used as a control. Surface regeneration was accomplished using 50 mM NaOH.

A series of three concentrations (duplicate points per sample) of either Fabs or mAbs were passed over the control or F-protein modified surfaces. Samples were diluted in running buffer to 1.25, 5.0, and 20.0 nM. A sample of running buffer only was included in the sample set as a negative control. The sensorgrams were analyzed using the evaluation software (BIAevaluation 3.2) provided by BIAcore. The background (buffer only) values were subtracted from the raw data, and background corrected data was imported into the BIAevaluation software. Using the simple dissociation model provided with the software, dissociation and association rate constants were determined (Langmuir binding model). These values were averaged and used to calculate the equilibrium dissociation constant (K_{D}).

As shown in Table 1, both the parental murine and chimeric 101F have approximately six-fold higher affinity than palivizumab, while the chimeric 101F Fab has similar affinity as the palivizumab Fab.

TABLE 1
Summary of BIAcore analyses binding for 101F and its
derivatives with immobilized RSV F-Protein

| | kₐₛₛ (M⁻¹s⁻¹) | k_{dis} (s⁻¹) | calc. K_{D} (pM) |
|---|---|---|---|
| palivizumab | 4.7 × 10⁵ | 5.0 × 10⁻⁴ | 1100 |
| palivizumab Fab | 7.2 × 10⁵ | 1.1 × 10⁻³ | 1500 |
| Murine 101F | 7.1 × 10⁵ | 1.4 × 10⁻⁴ | 190 |
| Chimeric human 101F | 8.9 × 10⁵ | 1.4 × 10⁻⁴ | 160 |
| Chimeric human 101F Fab | 7.4 × 10⁵ | 1.6 × 10⁻³ | 2100 |

### Example 8

### Virus Neutralization by Anti-RSV mAb 101F

HEp-2 cells (ATCC CCL-23) obtained from the American Tissue Type Collection (ATCC) (Manassas, Va.) were maintained in modified Eagle's media (MEM), supplemented with 10% fetal calf serum (heat-inactivated and gamma-irradiated, Hyclone, Logan, Utah), non-essential amino acids, penicillin G (100 units/ml) streptomycin (100 ug/ml) and 2 mM L-glutamine and grown at 37° C. in a humidified atmosphere of 5% CO₂. Human RSV (Long strain, ATCC VR-26) was obtained from the ATCC (Manassas, Va.). RSV stocks were prepared by infecting HEp-2 cells with RSV at a MOI of 0.01 PFU per cell. The culture supernatant was collected at 6 days post infection, adjusted to 10% sucrose and stored in liquid nitrogen. RSV titers were determined by plaque assay on HEp-2 cells using a 0.5% methylcellulose overlay in media. Antibody neutralization assays were performed in a 96-well format. Monoclonal antibodies were diluted in a 96-well plate starting from 6.4 ug/ml to 0.0125 ug/ml using two-fold dilutions in a 50 μl volume in media. Palivizumab (Synagis®, MedImmune Inc., Gaithersburg, Md.) was used as a positive control in all assays. An equal volume of 3×10⁴ PFU/ml RSV in media was added to each well. The mAb-virus mixtures were incubated at room temperature for two hours. Meanwhile, HEp-2 cells were seeded in a 96-well plate at a density of 1.5×10⁴/well in 100 microliters. After the one hour antibody incubation step, the mAb-virus mixture was added to the HEp-2 cells and incubated at 37° C. for various times and processed by one of the methods below. Six days post infection, the medium was removed and fifty μl of 0.5% crystal violet solution (prepared in 70% Methanol solution) was added to stain each well for 10 minutes as previously described (Trepanier et al., Virol. Methods, 1:343-347, 1980). Excess stain was removed and the fixed monolayers were gently washed with water, and then air-dried for 1 hour. One hundred μl of 70% methanol was then added to each well. After 10 minutes of gentle shaking, the optical density was measured at 570 nm using a microplate reader (Molecular Device, Menlo Park, Calif.). Prism 3.0 (Graphpad Software, Inc., San Diego, Calif.) was used for data analysis and to generate EC₅₀ curves.

As shown in Table 2, both the parental murine (hybridoma-derived and recombinantly generated) and the chimeric 101F have approximately the same (within 1-2 fold) virus neutralizing activity as palivizumab under the conditions described here. The Fabs derived from the chimeric 101F and palivizumab have approximately 10-fold and 16 to 30-fold lower activity than the parental IgGs, respectively.

TABLE 2
RSV neutralization activity of 101F

| | IC50 (ug/ml) |
|---|---|
| Palivizumab | 0.10 ± 0.06 |
| Palivizumab Fab | 3.2 ± 0.09 |
| Hybridoma-derived murine 101F | 0.19 ± 0.02 |
| Recombinant murine 101F | 0.09 ± 0.007 |
| Chimeric human 101F | 0.08 ± 0.02 |
| Chimeric human 101F Fab | 0.8 ± 0.04 |

* Data compiled from multiple assays

### Example 9

### Immunoprophylaxis in Cotton Rats

Antibodies were tested in an immunoprophylaxis model essentially as described by Prince et al., in Virus Res., 3:193-206, 1985. Inbred cotton rats (Sigmodon hispidus, average age 5 weeks, female gender weighing between 60-90 grams) were obtained from Harlan Sprague Dawley, Prattville, Ala. Weights of all animals used in a single study were within 3 grams of each other. On day 1, animals injected intramuscularly with various doses of mAbs or BSA (negative control). Twenty-four hours later, serum samples were taken, and animals were anesthetized with isoflurane, and then inoculated intranasally using a micropipet with RSV (10⁵ PFU virus) in a volume of 0.1 ml. Fours days after virus infection, animals were sacrificed by CO₂ asphyxiation, and lungs and serum were harvested. Serum samples were frozen at −80° C. Lungs from each animal were washed three times in ice-cold lung wash media (PBS containing 20 U/ml penicillin G, 20 ug/ml streptomycin, 100 ug/ml gentamicin and 0.25 ug/ml amphotericin B) and then minced by cutting with scissors, transferred to a Seward Stomacher® 80 Biomaster (Brinkmann Instruments, Inc., Westbury, N.Y.) and homogenized for two minutes at high speed. Viral titers were determined on HEp-2 cells and expressed as PFU/gm of lung tissue.

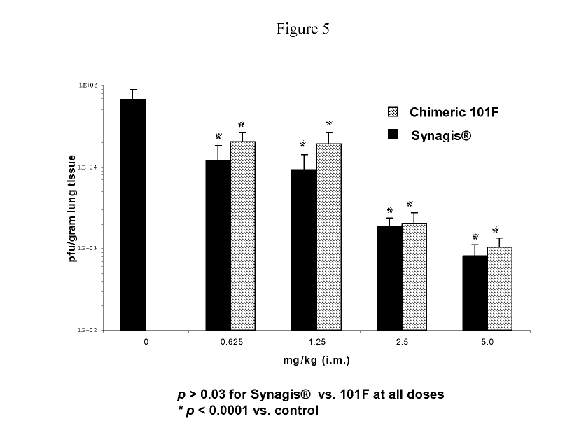

As shown in FIG. 5, both the chimeric 101F and palivizumab caused statistically significant reductions in viral lung titers at all doses tested, with a 99% reduction in viral lung titers at 5.0 mgs/kg.

### Example 10

Determination of Anti-RSV mAb 101F Epitope

A plasmid engineered to express the RSV F protein was constructed by first synthesizing the F gene of RSV A2 strain and 18537 strain with mammalian optimized codons for translation in eukaryotic cells and removal of all cryptic RNA processing signals similar to a previous report (Morton et al., Virology 311:275-288, 2003) (SEQ ID NOs: 46 and 47, respectively). These were cloned into a pcDNA 3.1 mammalian expression vector (Invitrogen, Inc.). Various mutations were made within the coding region of RSV F protein (A2 strain) which corresponded to previously described mAb escape mutants, in particular, Ser275 to Phe for palivizumab (Crowe et al., Virology, 252:373-375, 1998), Lys433 to Thr for mAb 7.936 (Lopez et al., J. Virol. 72:6922-6928, 1998), and Arg429 to Ser for mAb19 (Arbiza et al., J. Gen. Virol. 73:2225-2234, 1992). Plasmids expressing RSV F mutants were transfected into 293T cells. At 24 hours post transfection, cells were fixed with 0.05% glutaraldehyde in phosphate-buffered saline, and palivizumab or chimeric 101F binding was determined using an ELISA assay.

Table 3 shows the results indicated as mAb binding relative to wild-type RSV F protein. These results show that palivizumab and the chimeric 101F recognize distinct epitopes on RSV F protein. Furthermore, 101F is capable of binding to a mutant RSV F protein (Ser275Phe) that is no longer recognized by palivizumab.

TABLE 3
Comparison of 101F and palivizumab binding to point
mutants in the RSV F protein

| | Palivizumab | Chimeric 101F |
|---|---|---|
| Wild-type RSV F | 100% | 100% |
| S275F | 0% | 125% |
| R429S | 97% | 59.7% |
| K433T | 95.5% | 1% |

These data demonstrate that antibody 101F and palivizumab have similar potency in vitro and in vivo under these conditions. Furthermore, the 101F mAb recognizes a different epitope than palivizumab and is able to bind to mutants of RSV F that are no longer bound by palivizumab suggesting that 101F mapped to antigenic site IV,V,VI. Thus, 101F is expected to be able to neutralize viruses that are resistant to palivizumab.

The antigenic site IV,V,VI is complex and appears to contain overlapping epitopes as defined by several different mAbs such as mAb19 (Arbiza et al., J. Gen. Virol. 73:2225-2234, 1992; Lopez et al., J. Virol. 72:6922-6928, 1993). To genetically define the epitope, a panel of RSV F proteins containing single amino acid mutations in the site IV,V,VI region was expressed on the surface of mammalian cells by transient transfection and used to determine the binding of 101F, palivizumab, and mAb19 by ELISA. To ensure that any changes in mAb binding were not attributable to global changes in the F protein, each mutant was characterized with respect to level of expression, post-translational processing, cell surface expression and cell fusion activity.

The binding of 101F, mAb19, and palivizumab mAbs to wild-type and mutant RSV F proteins was assayed by ELISA using 293T cells transiently transfected with plasmids expressing either the wild-type RSV F proteins or a panel of RSV F mutants. Results are shown in Table 4 as binding of mAbs to the various point mutations is represented relative to binding to wild type RSV F protein. As chimeric 101F and palivizumab are both human IgG1kappa antibodies the same secondary antibody and detection reagents could be used allowing for a direct comparison of 101F and palivizumab binding.

The results indicate that both palivizumab, mAb19, and 101F bind to the wild-type RSV F protein from both subgroups (A2 strain and 18537 strain) equivalently. Palivizumab binding to S275F is completely eliminated while 101F and mAb19 binds S275F at a level similar to wild type. Surprisingly, 101F binding is only slightly reduced to R429S yet significantly reduced with K433T, while palivizumab binds both at a level similar to wild type. The significant decrease in 101F binding at residue 433 indicated this to be a critical residue for 101F interaction with RSV-F.

Screening of additional mutants confirmed that residue K433 is critical for 101F binding to RSV-F protein. Table 4 shows equivalent binding of 101F and palivizumab to K427D, K427Q, N428Q, R429K and I431A when compared to RSV-F wild type. 101F binding is reduced at N428D by ˜25% and at R429S and G430A by about 50% when compared to wild type. 101F binding is eliminated with K433L, K433N and K433T, and reduced at K433Q, K433R and K433S. Binding for both 101F and palivizumab at K433D is drastically reduced. This appears to be due to inefficient processing and cleavage of this particular mutant. A metabolic labeling assay indicated that although K433D is processed from F₀ to F₁ and F₂, it is to a lesser extant than all of the other mutants (data not shown). Table 4 also shows additional mutants at T434, S436, N437, S438 and V447. Binding of both 101F and palivizumab appear to be equivalent with these changes. This table also shows a double mutation containing both the palivizumab and mAb19 escape variants, S275F and R429S, respectively. Similar to the effect of the single S275F change, binding of palivizumab is abolished while binding of 101F is reduced by about 70%. Plasmid Delta R429-G466 contains an in frame deletion and results in defective processing of F₀ to F₁ and F₂, eliminating binding of both 101F and palivizumab. Taken together, these data indicate that residue K433 is critical for 101F binding. It also suggests that the mAb 19 escape variant R429S, although important for 101F binding, is not solely responsible for 101F binding.

TABLE 4
ELISA analysis of mAb binding to mammalian expressed
RSV-F protein.

| | % mAb binding relative to wild-type | | |
|---|---|---|---|
| RSV F point | Chimeric | Palivizumab | |
| mutants | 101F | (palivizumab) | mAb19 |
| Wild-type | 99.9 | 100.0 | 99.9 |
| (subgroup A, A2 | | | |
| strain) | | | |
| Wild-type | 91.6 | 132.5 | 105.2 |
| (subgroup B, | | | |
| 18537 strain) | | | |
| K272M | 126.6 | 3.8 | 100.5 |
| K272N | 95.5 | 15.3 | 62.3 |
| K272Q | 85.3 | 30.1 | 88.7 |
| K272T | 70.4 | 9.1 | 68.0 |
| S275F | 29.4 | 10.9 | 31.7 |
| T400A | 155.2 | 126.6 | 161.2 |
| C422S | 159.6 | 177.5 | 193.9 |
| K427D | 12.7 | 14.2 | −12.7 |
| K427E | 48.0 | 56.5 | 68.7 |
| K427Q | 94.9 | 135.7 | 154.9 |
| N428D | 116.2 | 139.4 | 121.3 |
| N428Q | 117.2 | 190.2 | 193.8 |
| R429K | 44.6 | 88.8 | 11.2 |
| R429S | 72.5 | 150.0 | 3.9 |
| G430A | 79.7 | 166.5 | 0.15 |
| I431A | 61.9 | 108.2 | 81.8 |
| I431L | 78.5 | 80.9 | 65.5 |
| I432L | 74.9 | 68.9 | 64.4 |
| I432Q | 49.3 | 60.8 | 57.9 |
| I432T | 191.7 | 206.9 | 169.8 |
| K433D | 1.1 | 29.9 | 1.6 |
| K433L | 3.8 | 88.1 | 24.5 |
| K433N | 2.9 | 80.7 | 28.7 |
| K433Q | 2.7 | 60.7 | 47.4 |
| K433R | −0.6 | 15.8 | 23.1 |
| K433T | 3.9 | 64.5 | 64.4 |
| K433S | 69.5 | 86.3 | 111.8 |
| T434S | 62.8 | 48.3 | 84.9 |
| F435Y | 27.3 | 21.7 | 9.2 |
| S436F | 151.2 | 85.3 | 163.3 |
| S436T | 74.1 | 119.6 | 85.8 |
| N437D | 30.8 | 83.3 | 33.2 |
| G438A | 9.2 | 50.7 | 19.0 |
| V447A | 71.4 | 138.8 | 62.7 |
| F483Y | 140.4 | 171.1 | 140.8 |
| F483L | 104.8 | 123.5 | 100.7 |
| D486N | 19.2 | 40.3 | 30.7 |
| F488Y | 41.1 | 79.8 | 56.0 |
| F488L | 19.0 | 60.3 | 40.0 |
| S275F + R429S | 33.1 | 2.1 | not tested |
| Deletion 429-446 | 5.2 | 3.2 | not tested |

To rule out the possibility that the changes in the level of mAb binding were due to more global alterations in the RSV F protein, the effect of the point mutations on expression, processing, and cell-surface levels of the RSV F protein was examined by immunoprecipitation and flow cytometry as described for wild-type RSV F. Transfected cells were labeled with [³⁵S]-methionine/cysteine and immunoprecipitated.

The results (data not shown) indicated that all mutant RSV-F plasmid DNA constructs express protein that is processed in a manner similar to RSV-F wild type, with the exception of K433D, which exhibited some reduction in the processing of F0 to F1 and F2, although F1 and F2 were still detectable. The relative expression levels of the mutant constructs appear to be similar to the wild type RSV-F, indicating that the specific point mutations do not have a marked effect on expression or protein processing. Additional controls included 293T cells infected with RSV virus (24 hrs post infection at a multiplicity of infection of 0.1 with Long strain). A mutant containing an in-frame deletion of 37 amino acids (deletion R429-G466) was included as a negative control for RSV F protein processing. This mutation results in expression of F0, but prevents processing to F1 and F2.

To confirm cell surface expression of the panel of RSV-F mutants, and to confirm the ELISA binding results, flow cytometry was used. Representative dot plots of 101F and palivizumab showed binding to RSV-F mutant containing specific point mutations R429S, I432T and K433T (data not shown). Binding of 101F was moderately reduced with the R429S mutation and almost completely eliminated with the K433T mutation in agreement with the ELISA results. These data confirm the cell surface expression of the recombinantly expressed RSV-F mutants and delineates differences in the relative binding of 101F and palivizumab to specific point mutations.

To biochemically characterize the 101F epitope, a trypsin digestion of a purified RSV F protein-101F mAb complex was performed, followed by mass spectrometry analysis of the resulting recovered mAb bound peptide. Based upon the sequence of the recovered peptide, a series of peptides deleted from the N-terminus and C-terminus further delineated the 101F epitope.

One peptide at m/z=3330 was captured by the chimeric 101F mAb. The same peptide was captured using Lys C as the digesting protease. Sequence assignment was based upon mass and matched with the database from a virtual trypsin digestion of the RSV F protein. This peptide, m/z=3330, was identified as residues 420-445 of the RSV F protein and has the sequence ₄₂₀TKCTASNKNRGIIKTFSNGCDYVSNK₄₄₅ (SEQ ID NO: 28).

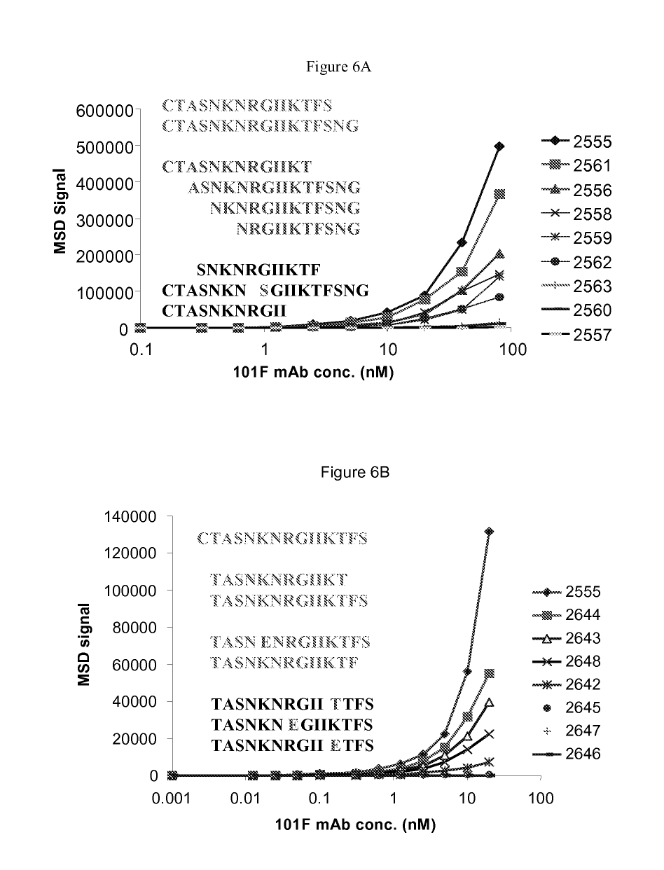

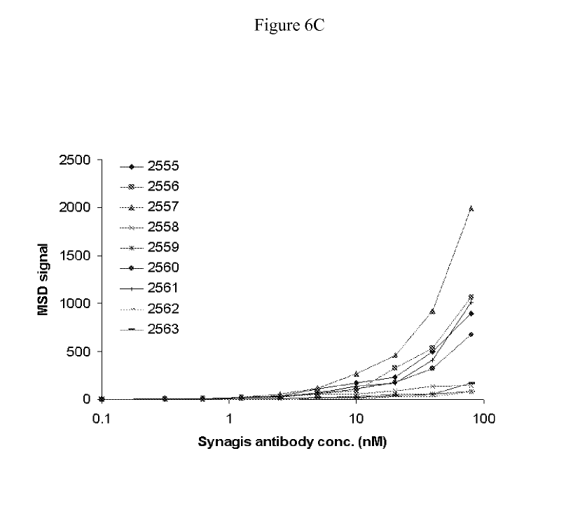

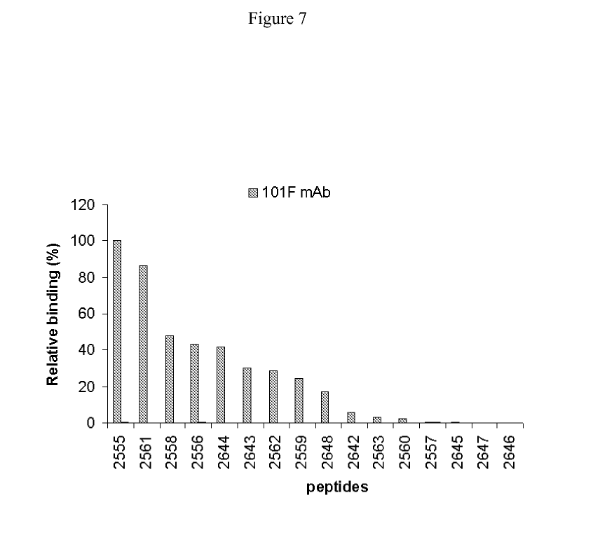

Sixteen N-terminal biotinylated synthetic peptides identified as CEN nos. 2555, 2561, 2556, 2558, 2559, 2562, 2563, 2560, 2557, 2555, 2644, 2643, 2648, 2642, 2645, 2647 and 2646, (SEQ ID NOs: 29 to 45, respectively) were incubated with MSD streptavidin-coated ELISA plates. 25 μl of 100 μg/ml of each peptide was added to a well and serial dilutions of either 101 F mAb or palivizumab were dispensed into each well. The binding results are shown in FIG. 6. The RSV F protein peptide, ₄₂₂CTASNKNRGIIKTFS₄₃₆ (SEQ ID NO: 29 and 38 (CEN2555)) was bound by chimeric 101F mAb with the highest affinity, compared to the other fifteen synthetic peptides (FIGS. 6A and 6B). Peptides nos. 2563, 2560, 2557, 2645, 2647 and 2646 had significantly reduced binding to chimeric 101F. Palivizumab bound these 16 peptides non-specifically (FIG. 6C) relative to the signal detected using chimeric 101F. To summarize and prioritize the binding affinity of chimeric 101F mAb to different synthetic peptides, a bar graph is shown in FIG. 7. In summary, the binding epitope determined by affinity-based protease digest and mass spectrometry is located in the region, ₄₂₀TKCTASNKNRGIIKTFSNGCDYVSNK₄₄₅ (SEQ ID NO: 28).

Further analysis using synthetic peptides refined the epitope. Based on the peptide ELISA data, the chimeric 101F-binding region could be reduced to ₄₂₂CTASNKNRGIIKTFS₄₃₆ (SEQ ID NO: 38). In addition, R429 and K433 significantly contribute to epitope binding.

This observation was confirmed by further substitution of R429 to S or E, and substitution of K433 to T and E using synthetic peptides. Another positively charged residue K427 makes a minor contribution to the binding as demonstrated by the substitution of K427 to E (FIG. 6).

Protease digestion of the mAb-antigen complex, ELISA based binding of F derived peptides, and a genetic analysis of a panel of RSV F mutants identified the same region of RSV F protein as being critical for the binding of 101F and show that this epitope is distinct from the epitope for palivizumab and mAb19. Residue K433 of the RSV-F protein is critical for 101F binding as demonstrated from data derived from a panel of qualitative and functional assays surveying protein processing and cleavage, cell surface expression, antibody binding, and peptide binding. As these peptides are recognized by a potent, broadly neutralizing RSV mAb (101F), immunization using these peptides, protein fusions containing these peptide sequences, or nucleic acids encoding these peptides and protein fusions containing these peptide sequences could be used as vaccines and elicit broadly reactive and potent serum antibodies against RSV.

### Example 11

### Generation of a Human-Adapted Anti-RSV mAb

The amino acid sequence of anti-RSV mAb 101F was used to query a human antibody database compiled from public antibody sequence databases. The variable region of the heavy chain of 101F (SEQ ID NO: 4) showed high homology to a vb_{—}2-05 heavy chain germline sequence of the human VH2 heavy chain family (SEQ ID NO: 48). A construct in which the CDR regions of 101F heavy chain were then transferred into a vb 2-05 related heavy chain sequence was synthesized to generate a human-adapted anti-RSV mAb heavy chain B21M having the variable region amino acid sequence shown in SEQ ID NO: 49 and FIG. 2c.

The mAb 101F V kappa light chain amino acid sequence (SEQ ID NO: 6) of 101F showed the greatest homology to the vb B3 light chain V Kappa germline sequence of the human VK IV family (SEQ ID NO: 50). The CDR regions of 101F were transferred into this backbone to generate the B21M Vk light chain whose variable region sequence is shown in SEQ ID NO: 51 and FIG. 2d.

### Example 12

### Generation and Characterization of B21M Heavy and Light Chain Variants

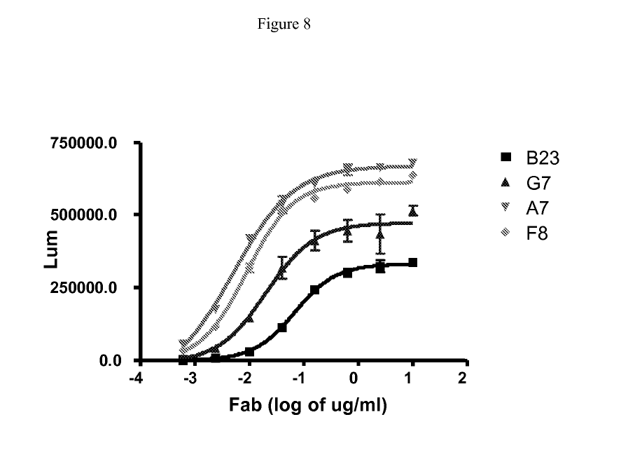

Fab libraries of CDR variants from B21M were prepared in a pIX phage display system by saturation mutagenesis of selected residues in Lc-CDR1 and Lc-CDR3 and Hc-CDR2 and Hc-CDR3. Purified Fabs (>90% purity and codon optimized for E. coli production) were tested for binding to the human RSV F protein by ELISA (as in Example 6 above) and CDR mutations that resulted in improved binding over wild type were identified. Representative ELISA data is shown in FIG. 8. These results shown a significant improvement in the binding affinity of clones G7, A7 and F8 over wild-type B23M antibody.

The binding association (Kₐ), dissociation constant (K_{d}) as well as binding affinity (K_{D}) were determined by BIAcore analysis on the purified Fabs and confirmed the ELISA results. The Fab/RSV-F binding assays were performed at 25° C. using either Biacore 2000 or 3000 biosensors equipped with a CM5 (carboxymethyl dextran) chip. All surfaces (flow cells) were modified by immobilizing 500-1000 RU SA (streptavidin) at 10 μg/mL in 10 mM sodium acetate, pH 5.0 using a standard amine coupling method. The sample and running buffer were PBS with 0.1% P-20 added to minimize non-specific binding of sample contaminants to the dextran surface. A recombinant RSV-F was biotinylated and captured on three SA surfaces on the sensor chip. About 40 RU biotinylated RSV-F were captured on flow cells 1, 25 RU on flow cell 3. Flow cell 2 was used as a reference surface where no F protein was used. Fab samples were injected at one concentration (30 nM) over the four surfaces. The association phases were monitored for 2 minutes and dissociation phases for 6 to 60 minutes. The longer dissociation times were required to measure accurately complex decay of tight interactions. The RSV-F surfaces were regenerated with a 3-second pulse 10 mM phosphoric acid at the end of each binding cycle. Each sample analysis was repeated three times. Fab B23 was used as an activity reference. Samples of Fab 23 were injected at 0, 1.23, 3.7, 11.1, 33.3, 100, and 300 nM.

The Fab/RSV-F binding data from flow cells 1 and 3 were corrected using the reference data obtained from the flow cell 2. The resulting corrected binding response data was fit to a 1:1 interaction model using the CLAMP™ software. The binding rate constants were directly obtained from the fit to the 1:1 model to the replicate data set. The equilibrium binding constant was calculated from their ratio (K_{D}=k_{d}/kₐ).

Fabs with improved binding activity to human RSV F protein relative to B21M were also sequenced. Table 5 summarizes the binding characteristics of the Fabs as determined by BIAcore analysis and their CDR amino acid changes. The data for the wild type Fab B21M is listed at the top of Table 5. The SEQ ID NOs of the complete amino acid sequences of the V_{L} and V_{H} regions containing the CDR variants are also listed in the first column of Table 5; the first SEQ ID NO: represents the V_{L} sequence, the second the V_{H} sequence.

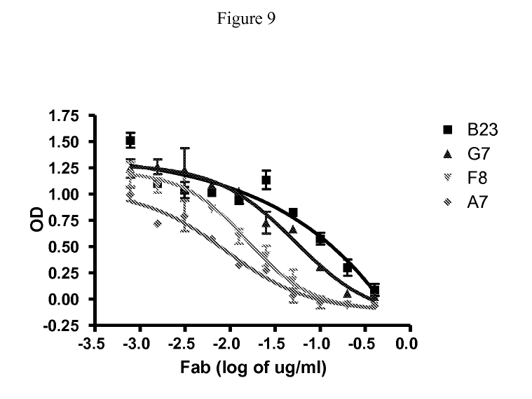

The results indicate that mutations that resulted in binding improvements over 10-fold were in Lc-CDR1. The amino acid sequences of the Lc-CDR1 variants A7, H8, F8, A2, G3, F5, A10, H4, C11, B8, A11 and B6 are shown in SEQ ID NOs: 52, 53, 54, 55, 56, 57, 105, 58, 59, 60, 61 and 62, respectively. For example, the Y31L/N32F/I34R mutation in Lc-CDR1 (clone A7, SEQ ID NO: 52) showed 32-fold binding improvement and a 10-fold decrease in anti-viral IC₅₀. Fab 009 that combined the mutations of A7 with additional Lc CDR mutations, i.e., Q27D and E97D, resulted in a further decrease, relative to A7, in binding dissociation constant (K_{d}) of approximately 4 fold. Mutations in other CDRs, i.e, Fab 004 (Hc-CDR3 mutations) and Fab G7 (Lc-CDR3 mutation) also resulted in incremental binding improvement over the wild type Fab. Correlated to binding affinity improvement, the Fabs showed improved anti-viral activity in an in vitro RSV micro-neutralization assay conducted as described in Example 8 above (data shown in FIG. 9).

The sequence results revealed significant selection of a set of changes in the designed Lc-CDR region, present in those Fabs with improved binding activity. For example, phenylalanine, an aromatic amino acid, was predominantly selected over the wild type asparagines and 18 other possible amino acids at position 32. At position 34, a charged or a polar amino acid is preferred over the wild type isoleucine, in combination with the phenylalanine change at position 32. Amino acid substitutions at position 31 were conservative, suggesting the wild type or related amino acids were preferred. The wild type amino acid tyrosine at position 36 was selected in these variants.

The ability of the Fabs listed in Table 5 to neutralize RSV virus was determined as in Example 8 above. The data is reported in Table 5 as virus neutralization fold increase over wild-type B23 Fab (E. coli codon optimized B21M Fab). It is expected that whole antibody prepared from the Fabs described in Table 5 would exhibit similar antigen binding and virus neutralization activities.

TABLE 5
B21M CDR Variant Fabs Sequence and Activity

| | | | | | | | | | | | | | Activity | | | |
|---|---|---|---|---|---|---|---|---|---|---|---|---|---|---|---|---|
| SEQ | | | Sequence | | | | | | | | | | Biacore (Fab) | | | |
| ID | Clone | Hc-CDR2 | | | Hc-CDR3 | | Lc-CDR1 | | | | | Lc- CDR3 | Ka | Kd | KD | |
| NO. | AbPD | I53 | K59 | N62 | Y101 | T104 | Q27 | Y31 | N32 | I34 | M37 | E97 | (×10⁶ M^{-1s-1}) | (×10⁻⁴s⁻¹) | (pM) | Viral Neut |
| | B21M Fab | — | — | — | — | — | — | — | — | — | — | — | 0.65 | 10 | 1580 | |
| 63, 49 | A7 | — | — | — | — | — | — | L | F | R | — | — | 2.42 | 0.87 | 36 | 23X |
| 64, 49 | H8 | — | — | — | — | — | — | L | F | K | — | — | 4.19 | 0.95 | 23 | 22X |
| 65, 49 | F8 | — | — | — | — | — | — | L | F | Q | — | — | 1.66 | 0.86 | 52 | 6-7X |
| 66, 49 | A2 | — | — | — | — | — | — | L | F | V | — | — | 2.50 | 2.2 | 87 | 7.4X |
| 67, 49 | G3 | — | — | — | — | — | — | H | F | K | V | — | 2.10 | 2.3 | 110 | 8.6X |
| 68, 49 | F5 | — | — | — | — | — | — | V | F | R | — | — | 1.40 | 2.4 | 170 | 4X |
| 104, 49 | A10 | — | — | — | — | — | — | L | L | G | — | — | 0.85 | 1.47 | 175 | ND |
| 69, 49 | H4 | — | — | — | — | — | — | L | F | T | — | — | 2.02 | 0.87 | 43 | <12X |
| 70, 49 | C11 | — | — | — | — | — | — | — | L | R | — | — | 1.40 | 2 | 138 | 11X |
| 71, 49 | B8 | — | — | — | — | — | — | L | F | S | — | — | 1.24 | 1.17 | 94 | 4X |
| 72, 49 | A11 | — | — | — | — | — | — | — | S | R | — | — | 1.39 | 2.34 | 168 | 3X |
| 73, 49 | B6 | — | — | — | — | — | — | F | F | K | — | — | 2.04 | 0.81 | 40 | 12X |
| 106, 74 | G7 | L | — | — | — | — | — | — | — | — | — | D | 2.00 | 2.40 | 121 | 14-16X |
| 51, 75 | C7-P2 | — | — | — | W | R | — | — | — | — | — | — | 0.72 | 8.7 | 1220 | <1X |
| 51, 76 | B6-P2 | — | — | — | W | K | — | — | — | — | — | — | 0.65 | 3.3 | 510 | 2X |
| 51, 77 | C8-P2 | — | — | — | W | A | — | — | — | — | — | — | 0.80 | 0.9 | 110 | 2X |
| 51, 78 | A5-P2 | — | — | — | — | R | — | — | — | — | — | — | 0.94 | 6.2 | 650 | <1X |
| 51, 79 | E3-P2 | — | — | — | — | K | — | — | — | — | — | — | 1.26 | 5.82 | 560 | <1X |
| 51, 80 | B10-P2 | — | — | — | — | A | — | — | — | — | — | — | 0.59 | 4.85 | 820 | 5X |
| 76, 63 | 004 | — | — | — | W | K | — | L | F | R | — | — | 1.61 | 0.46 | 28 | 4X |
| 63, 80 | 005 | — | — | — | — | A | — | L | F | R | — | — | 1.80 | 1.06 | 59 | 8X |
| 63, 79 | 006 | — | — | — | — | K | — | L | F | R | — | — | 2.30 | 1.6 | 69 | 3X |
| 81, 49 | 007 | — | — | — | — | — | — | L | F | K | — | D | 3.30 | 1.9 | 58 | 5X |
| 82, 49 | 008 | — | — | — | — | — | D | L | F | K | — | D | 2.10 | 2.8 | 133 | 15X |
| 83, 49 | 009 | — | — | — | — | — | D | L | F | R | — | D | 2.80 | 0.79 | 28 | 19X |
| 83, 84 | 010 | L | Y | — | — | — | D | L | F | R | — | D | 3.8 | 1.8 | 47 | 18X |
| 85, 86 | 011 | L | — | — | — | — | D | L | F | Q | — | D | 4.0 | 0.27 | 7 | >23X |
| 81, 86 | 012 | L | — | — | — | — | — | L | F | K | — | D | 3.0 | 0.015 | 5 | 11X |
| 85, 84 | 013 | L | Y | — | — | — | D | L | F | Q | — | D | 3.6 | 2.10 | 58 | 13X |
| 87, 88 | 014 | — | — | S | — | — | D | L | F | V | — | D | 0.38 | 0.70 | 184 | <1X |
| 89, 86 | 015 | L | — | — | — | — | — | F | F | T | — | D | 5.0 | 0.78 | 16 | 8X |
| 99, 88 | 016 | — | — | S | — | — | — | — | F | V | — | D | 1.0 | 0.90 | 90 | 10X |
| 108, 88 | 017 | — | — | S | — | — | D | — | F | Q | — | D | 1.5 | 0.81 | 54 | >23X |
| 90, 88 | 018 | — | — | S | — | — | D | L | F | Q | — | — | 1.6 | 0.88 | 55 | 11X |
| 85, 88 | 019 | — | — | S | — | — | D | L | F | Q | — | —D | 3.34 | 0.45 | 135 | >23X |
| 109, 88 | 020 | — | — | S | — | — | D | L | F | Q | — | A | ND | ND | ND | 8X |
| 95, 88 | 022 | — | — | S | — | — | — | L | F | T | — | D | 2.16 | 0.54 | 25 | 22X |
| 94, 86 | 024 | L | — | — | — | — | D | L | F | T | — | D | 3.14 | 0.07 | 2 | >23X |
| 81, 88 | 025 | — | — | S | — | — | — | L | F | K | — | D | 2.52 | 0.46 | 18 | 20X |
| 97, 86 | 026 | L | — | — | — | — | D | F | F | K | — | D | 2.39 | 0.66 | 28 | 20X |
| 111, 49 | 028 | — | — | — | — | — | D | L | F | V | — | — | 4.2 | 0.86 | 20 | 18X |
| 100, 88 | 029 | — | — | S | — | — | D | — | F | V | — | D | 4.58 | 0.04 | 9 | >23X |
| 101, 88 | 030 | — | — | S | — | — | — | — | L | K | — | D | 1.03 | 0.75 | 73 | 18X |
| 63, 77 | 031 | — | — | — | W | A | — | L | F | R | — | — | 2.53 | 0.57 | 23 | 8X |
| 112, 88 | 032 | — | — | S | — | — | D | — | F | R | — | D | 0.93 | 0.36 | 39 | >23X |
| 112, 49 | 033 | — | — | — | — | — | D | — | F | R | — | D | ND | ND | ND | ND |
| 103, 86 | 034 | L | — | — | — | — | — | — | F | T | — | D | ND | ND | ND | ND |
| 103, 88 | 035 | — | — | S | — | — | — | — | F | T | — | D | ND | ND | ND | ND |
| 63, 88 | F-1 | — | — | S | — | — | — | L | F | R | — | — | ND | ND | ND | ND |
| 85, 93 | F-2 | L | — | S | — | — | D | L | F | Q | — | D | ND | ND | ND | ND |
| 94, 93 | F-3 | L | — | S | — | — | D | L | F | T | — | D | ND | ND | ND | ND |

Note:
“—” indicate wild type sequence;
ND = not determined

### Example 13

### Generation and Characterization of Polyethylene Glycol Modified Anti-RSV mAbs

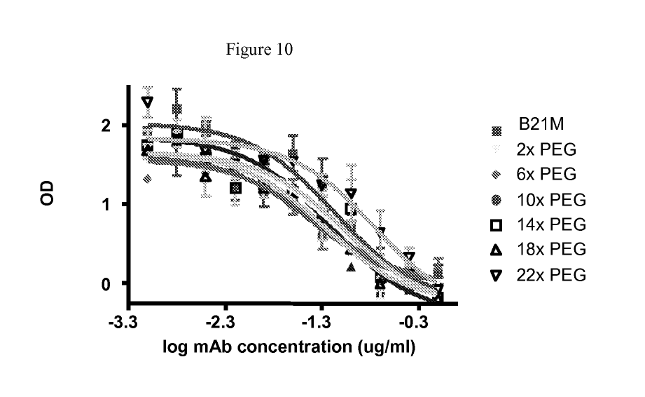

Purified mAb (B21M) was polyethylene glycol modified (PEGylated) using commercially available reagents from Nektar Therapeutics, San Carlos, Calif. (Cat No. 2M4M0P01). While generally done site specifically, it has been demonstrated that a random coupling through the amine groups produces active PEGylated mAb despite the presence of lysines in the CDRs (data not shown). Although not theoretically limited to this range, mAb:PEG ratios from 1:1 to 1:24 were tested. SDS PAGE indicated that, over this range, the bulk of the material was PEGylated 0-6 times with higher concentrations modifying more starting material although starting material was never fully modified (data not shown). Additional quenched PEG was added to bring all the samples to the same PEG concentration before activity testing. PEGylated samples were tested for antiviral activity using a standard neutralization assay. The results are shown in FIG. 10 and indicate that PEGylation has no adverse effect upon the antiviral activity of this antibody. It is expected that PEGylation of whole antibody containing the CDR variants of Table 5 would have no adverse effect on antibody function and would result in increased half life of these molecules. The present invention now being fully described, it will be apparent to one of ordinary skill in the art that many changes and modifications can be made thereto without departing from the spirit or scope of the appended claims.

1. An isolated antibody reactive with Respiratory Syncytial Virus (RSV) F protein having the antigen binding ability of a monoclonal antibody having the amino acid sequences of the heavy chain complementarity determining regions (CDRs) as set forth in SEQ ID NOs: 8, 10 and 12 and the amino acid sequences of the light chain CDRs as set forth in SEQ ID NOs: 14, 16 and 18.

2. An isolated antibody reactive with RSV isolates that escape neutralization by antibodies or antibody fragments containing CDRs derived from palivizumab or mAb19.

3. The isolated antibody of claim 2 wherein the RSV isolate comprises a Ser275Phe mutation in the F protein.

4. An isolated antibody reactive with an RSV F protein epitope located at residues 422 to 436 (SEQ ID NO: 28) of the F protein.

5. An isolated antibody reactive with residues R429 and K433 of an RSV F protein.

6. The isolated antibody of claim 1 comprising the heavy chain CDR1 (Hc-CDR1), CDR2 (Hc-CDR2) and CDR3 (Hc-CDR3) amino acid sequences of SEQ ID NOs: 8, 10 and 12, respectively and the light chain CDR1 (Lc-CDR1), CDR2 (Lc-CDR2) and CDR3 (Lc-CDR3) amino acid sequences of SEQ ID NOs: 14, 16 and 18, respectively.

7. The isolated antibody of claim 1 comprising a variable region heavy chain (V_{H}) having the amino acid sequence of SEQ ID NO: 3 and a variable region light chain (V_{L}) having the amino acid sequence of SEQ ID NO: 5.

8. The isolated antibody of claim 1 comprising a V_{H} having the amino acid sequence of SEQ ID NO: 49 and a V_{L} having the amino acid sequence of SEQ ID NO: 51.

9. An isolated antibody having Hc-CDR1, Hc-CDR2 and Hc-CDR3 amino acid sequences as shown in SEQ ID NOs: 8, 10 and 12, respectively and a Lc-CDR1 as shown in Formula (I):
(I)
Arg Ala Ser Xaa₀ Ser Val Asp Xaa₁ Xaa₂ Gly Xaa₃
Ser Tyr Xaa₄ His
wherein Xaa₀ is Gln, Asp or His; Xaa₁ is Leu, His, Val, Phe or Tyr; Xaa₂ is Phe, Leu or Ser; Xaa₃ is Arg, Lys, Gln, Val, Gly, Thr or Ser; and Xaa₄ is Val or Met; and light chain CDR2 (Lc-CDR2) and light chain CDR3 (Lc-CDR3) amino acid sequences as shown in SEQ ID NOs: 16 and 18, respectively.

10. The isolated antibody of claim 9 comprising a V_{H} having the amino acid sequence of SEQ ID NO: 49 and a V_{L} having the amino acid sequence of SEQ ID NO: 63, 64, 65, 66, 67, 68, 105, 69, 70, 71, 72, 73 or 111.

11. An isolated antibody having a Hc-CDR1 amino acid sequence as shown in SEQ ID NO: 8, a Hc-CDR2 amino acid sequence as shown in Formula (II):
(II)
His Xaa₇ Tyr Trp Asp Asp Asp Xaa₈ Arg Tyr Xaa₉ Pro
Ser Leu Lys Ser
wherein Xaa₇ is Ile or Leu, Xaa₈ is Lys or Tyr and Xaa₉ is Asn or Ser, a Hc-CDR3 amino acid sequences as shown in SEQ ID NO: 12 and a Lc-CDR1 and Lc-CDR2 amino acid sequence as shown in SEQ ID NOs: 14 and 16, respectively and a Lc-CDR3 amino acid sequence as shown in SEQ ID NO: 107 or 110.

12. The isolated antibody of claim 11 comprising a V_{L} amino acid sequence as shown in SEQ ID NO: 74 and a V_{H} amino acid sequence as shown in SEQ ID NO: 106.

13. An isolated antibody having Hc-CDR1 and Hc-CDR2 amino acid sequences as shown in SEQ ID NOs: 8 and 10, respectively and a Hc-CDR3 amino acid sequence as shown in Formula (III):

| | |
|---|---|
| Leu Xaa₅ Gly Phe Xaa₆ Tyr Gly Phe Ala Tyr | (III) |

wherein Xaa₅ is Tyr or Trp; and Xaa₆ is Arg, Lys or Ala and Lc-CDR1, Lc-CDR2 and Lc-CDR3 amino acid sequences are as shown in SEQ ID NOs:
14, 16 and 18, respectively.

14. The isolated antibody of claim 13 comprising a V_{L} amino acid sequence as shown in SEQ ID NO: 51 and a V_{H} amino acid sequence as shown in SEQ ID NOs :75, 76, 77, 78, 79 or 80.

15. An isolated antibody having Hc-CDR1 and Hc-CDR2 amino acid sequences as shown in SEQ ID NOs: 8 and 10, respectively and a Hc-CDR3 amino acid sequence as shown in Formula (IV), a Lc-CDR1 amino acid sequence as shown in Formula (I) and Lc-CDR2 and Lc-CDR3 amino acid sequences as shown in SEQ ID NOs: 16 and 18, respectively.

16. The isolated antibody of claim 15 comprising a V_{L} amino acid sequence as shown in SEQ ID NO: 63 and a V_{H} amino acid sequence as shown in SEQ ID NOs: 76, 80 or 79.

17. An isolated antibody having a Hc-CDR1, Hc-CDR2 and Hc-CDR3 amino acid sequence as shown in SEQ ID NOs: 8, 10 and 12, respectively and a Lc-CDR1 amino acid sequence as shown in Formula (I) wherein Xaa₀ is Gln or Asp, Xaa₁ is Leu or Tyr, Xaa₂ is Phe, Xaa₃ is Lys or Arg and Xaa₄ is Met, a Lc-CDR2 amino acid sequence as shown in SEQ ID NO: 16 and a Lc-CDR3 amino acid sequence as shown in SEQ ID NO: 107.

18. The isolated antibody of claim 17 comprising a V_{L} amino acid sequence as shown in SEQ ID NOs: 81, 82, 83 or 112 and a V_{H} amino acid sequence as shown in SEQ ID NO: 49.

19. An isolated antibody having a Hc-CDR1 amino acid sequence as shown in SEQ ID NO: 8, a Hc-CDR2 of formula II, a Hc-CDR3 of formula III, a Lc-CDR1 of formula I, a Lc-CDR2 amino acid sequence as shown in SEQ ID NO: 16 and a Lc-CDR3 amino acid sequence as shown in SEQ ID NO: 107.

20. The isolated antibody of claim 19 comprising the following V_{L} and V_{H} amino acid sequence combinations:

| V_{L} SEQ ID NO: | V_{H} SEQ ID NO: |
|---|---|
| 83 | 84; |
| 85 | 86; |
| 81 | 86; |
| 85 | 84; |
| 87 | 88; |
| 89 | 86; |
| 85 | 88; |
| 91 | 86; |
| 92 | 93; |
| 94 | 86; |
| 95 | 88; |
| 96 | 88; |
| 97 | 86; |
| 81 | 88; |
| 99 | 88; |
| 87 | 49; |
| 100 | 88; |
| 101 | 88; |
| 102 | 88; |
| 102 | 49; |
| 103 | 86; |
| 103 | 88; |
| 108 | 88; |
| 90 | 88; |
| 85 | 88; |
| 109 | 88; |
| 63 | 88; |
| 85 | 93; |
| 94 | 93; or |
| 112 | 88. |

21. An isolated antibody having a Hc-CDR1 amino acid sequence as shown in SEQ ID NO: 8, a Hc-CDR2 of formula II, a Hc-CDR3 of formula III, a Lc-CDR1 of formula I, a Lc-CDR2 amino acid sequence as shown in SEQ ID NO: 16 and a Lc-CDR3 amino acid sequence as shown in SEQ ID NO: 18.

22. The isolated antibody of claim 20 comprising the following V_{L} and V_{H} amino acid sequence combinations:

| V_{L} SEQ ID NO: | V_{H} SEQ ID NO: |
|---|---|
| 90 | 86; |
| 90 | 88; |
| 98 | 49; or |
| 63 | 77 |

23. The isolated antibody of claim 1 wherein the antibody is of human origin.

24. The isolated antibody of claim 1 wherein the antibody is of murine origin.

25. The isolated antibody of claim 1 wherein the antibody comprises a Fab fragment.

26. The isolated antibody of claim 1 wherein the antibody comprises a scFv fragment.

27. The isolated antibody of claim 1 wherein the antibody or fragment is human-adapted.

28. The isolated antibody of claim 1 wherein the antibody or fragment comprises a chimeric antibody.

29. The isolated antibody of claim 1 wherein the antibody is conjugated to polyethylene glycol.

30. The isolated antibody of claim 1 wherein the antibody or fragment comprises murine antigen binding residues and human antibody residues.

31. A pharmaceutical composition comprising the isolated antibody of claim 1.

32. An isolated polynucleotide encoding the antibody of claim 1, 2, 4, 5, 9, 11, 13, 15, 17, 19 or 21.

33. A vector comprising the polynucleotide of claim 32.

34. A host cell comprising the vector of claim 33.

35. A method of making an anti-RSV antibody comprising culturing the host cell of claim 34 and recovering the monoclonal antibody produced by the host cell.

36. A hybridoma cell line that produces the antibody of claim 1.

37. A method of preventing RSV-induced disease comprising administering, to a patient at risk thereof, a prophylactically effective amount of the antibody of claim 1, 2, 4, 5, 9, 11, 13, 15, 17, 19 or 21.

38. A method of treating RSV-induced disease comprising administering to a patient a therapeutically effective amount of the antibody of claim 1, 2, 4, 5, 9, 11, 13, 15, 17, 19 or 21.

39. An isolated polypeptide comprising the peptide CTASNKNRGIIKTFS (SEQ ID NO: 38).

40. An isolated nucleic acid encoding the amino acid sequence of SEQ ID NO: 38.

41. A vaccine preparation comprising the isolated polypeptide of claim 39.

42. A vaccine preparation comprising the isolated nucleic acid of claim 40.

43. A method of immunizing a patient against RSV-induced disease by administering the vaccine preparation of claim 41 or 42.


SEQUENCE LISTING (112 sequences)

SEQ ID NO: 1 - DNA, 1722 bp, Respiratory Syncytial Virus
atggagctgctgatcctgaaggccaacgccatcaccaccatcctgaccgccgtgaccttctgcttcgccagcggccagaacatcaccgaggagttctaccagagcacctgcagcgccgtgagcaagggctacctgagcgccctgcgcaccggctggtacaccagcgtgatcaccatcgagctgagcaacatcaagaagaacaagtgcaacggcaccgacgccaaagtgaagctgatcaagcaggagctggacaagtacaagaacgccgtgaccgagctgcagctgctgatgcagagcacccaggccaccaacaaccgcgcccgccgcgagctgccccgcttcatgaactacaccctgaacaacgccaagaagaccaacgtgaccctgagcaagaagcgcaagcgccgcttcctgggcttcctgctgggcgtgggcagcgccatcgccagcggcgtggccgtgagcaaagtgctgcacctggagggcgaagtgaacaagatcaagagcgccctgctgagcaccaacaaggccgtggtgagcctgagcaacggcgtgagcgtgctgaccagcaaagtgctggacctgaagaactacatcgacaagcagctgctgcccatcgtgaacaagcagagctgcagcatcagcaacatcgagaccgtgatcgagttccagcagaagaacaaccgcctgctggagatcacccgcgagttcagcgtgaacgccggcgtgaccacccccgtgagcacctacatgctgaccaacagcgagctgctgagcctgatcaacgacatgcccatcaccaacgaccagaagaagctgatgagcaacaacgtgcagatcgtgcgccagcagagctacagcatcatgagcatcatcaaggaggaagtgctggcctacgtggtgcagctgcccctgtacggcgtgatcgacaccccctgctggaagctgcacaccagccccctgtgcaccaccaacaccaaggagggcagcaacatctgcctgacccgcaccgaccgcggctggtactgcgacaacgccggcagcgtgagcttcttcccccaggccgagacctgcaaagtgcagagcaaccgcgtgttctgcgacaccatgaacagcctgaccctgcccagcgaagtgaacctgtgcaacgtggacatcttcaaccccaagtacgactgcaagatcatgaccagcaagaccgacgtgagcagcagcgtgatcaccagcctgggcgccatcgtgagctgctacggcaagaccaagtgcaccgccagcaacaagaaccgcggcatcatcaagaccttcagcaacggctgcgactacgtgagcaacaagggcgtggacaccgtgagcgtgggcaacaccctgtactacgtgaacaagcaggagggcaagagcctgtacgtgaagggcgagcccatcatcaacttctacgaccccctggtgttccccagcgacgagttcgacgccagcatcagccaagtgaacgagaagatcaaccagagcctggccttcatccgcaagagcgacgagctgctgcacaacgtgaacgccggcaagagcaccaccaacatcatgatcaccaccatcatcatcgtgatcatcgtgatcctgctgagcctgatcgccgtgggcctgctgctgtactgcaaggcccgcagcacccccgtgaccctgagcaaggaccagctgagcggcatcaacaacatcgccttcagcaac

SEQ ID NO: 2 - protein, 574 aa, Respiratory Syncytial Virus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - DNA, 361 bp, Mus musculus
caggttactctgaaagagtctggccctgggatattgcagccctcccagaccctcagtctgacttgttctttctctgggttttcactgagcacttctggtatgggtgtgagctggattcgtcagccctcaggaaagggtctggagtggctggcacacatttactgggatgatgacaagcgctataacccatccctgaagagccgactcacaatctccaaggatacctccagaaaccaggtattcctcaagatcaccagtgtggacactgcagatactgccacatattactgtgctcgactctacggttttacctacggctttgcttactggggccaagggactctggtcactgtctctgcag

SEQ ID NO: 4 - protein, 120 aa, Mus musculus
QVTLKESGPGILQPSQTLSLTCSFSGFSLSTSGMGVSWIRQPSGKGLEWLAHIYWDDDKRYNPSLKSRLTISKDTSRNQVFLKITSVDTADTATYYCARLYGFTYGFAYWGQGTLVTVSA

SEQ ID NO: 5 - DNA, 336 bp, Mus musculus
gacattgtgctgacccaatctccagcttctttggctgtgtctctaggacagagagccactatcttctgcagagccagccagagtgtcgattataatggaattagttatatgcactggttccaacagaaaccaggacagccacccaaactcctcatctatgctgcatccaacccagaatctgggatccctgccaggttcactggcagtgggtctgggacagacttcaccctcaacatccatcctgtggaggaggaagatgctgcaacctattactgtcagcaaattattgaggatccgtggacgttcggtggaggcaccaagctggaaatcaaacgg

SEQ ID NO: 6 - protein, 111 aa, Mus musculus
DIVLTQSPASLAVSLGQRATIFCRASQSVDYNGISYMHWFQQKPGQPPKLLIYAASNPESGIPARFTGSGSGTDFTLNIHPVEEEDAATYYCQQIIEDPWTFGGGTKLEIK

SEQ ID NO: 7 - DNA, 21 bp, Mus musculus
acttctggtatgggtgtgagc

SEQ ID NO: 8 - protein, 7 aa, Mus musculus
TSGMGVS

SEQ ID NO: 9 - DNA, 48 bp, Mus musculus
cacatttactgggatgatgacaagcgctataacccatccctgaagagc

SEQ ID NO: 10 - protein, 16 aa, Mus musculus
HIYWDDDKRYNPSLKS

SEQ ID NO: 11 - DNA, 30 bp, Mus musculus
ctctacggttttacctacggctttgcttac

SEQ ID NO: 12 - protein, 10 aa, Mus musculus
LYGFTYGFAY

SEQ ID NO: 13 - DNA, 45 bp, Mus musculus
agagccagccagagtgtcgattataatggaattagttatatgcac

SEQ ID NO: 14 - protein, 15 aa, Mus musculus
RASQSVDYNGISYMH

SEQ ID NO: 15 - DNA, 21 bp, Mus musculus
gctgcatccaacccagaatct

SEQ ID NO: 16 - protein, 7 aa, Mus musculus
AASNPES

SEQ ID NO: 17 - DNA, 27 bp, Mus musculus
cagcaaattattgaggatccgtggacg

SEQ ID NO: 18 - protein, 9 aa, Mus musculus
QQIIEDPWT

SEQ ID NO: 19 - DNA, 23 bp, Artificial Sequence
  Primer
cgactggagcacgaggacactga

SEQ ID NO: 20 - DNA, 19 bp, Artificial Sequence
  Primer
ctgtcccgaggtctcaagg

SEQ ID NO: 21 - DNA, 20 bp, Artificial Sequence
  Primer
gaactgtgactacagagacc

SEQ ID NO: 22 - DNA, 33 bp, Artificial Sequence
  Primer
ttcgtacggccaccatggacaggcttacttcct

SEQ ID NO: 23 - DNA, 30 bp, Artificial Sequence
  Primer
ttcgaaacttacctgcagagacagtgacca

SEQ ID NO: 24 - DNA, 41 bp, Artificial Sequence
  Primer
ggtgcgtccttcgaccaccatggagtcagacacactcctgc

SEQ ID NO: 25 - DNA, 41 bp, Artificial Sequence
  Primer
cgcctccgcttcgacttacgtttgatttccagcttggtgcc

SEQ ID NO: 26 - DNA, 42 bp, Artificial Sequence
  Primer
ggtgcgtccttcgaccaccatggacaggcttacttcctcatt

SEQ ID NO: 27 - DNA, 44 bp, Artificial Sequence
  Primer
cgcctccgcttcgacttacctgcagagacagtgaccagagtccc

SEQ ID NO: 28 - protein, 26 aa, Respiratory Syncitial Virus
  Peptide Chain
TKCTASNKNRGIIKTFSNGCDYVSNK

SEQ ID NO: 29 - protein, 15 aa, Respiratory Syncitial Virus
  Peptide Chain
CTASNKNRGIIKTFS

SEQ ID NO: 30 - protein, 16 aa, Artificial Sequence
  Respiratory Syncitial Virus
CTASNKNRGIIKTFSN

SEQ ID NO: 31 - protein, 13 aa, Artificial Sequence
  Respiratory Syncitial Virus
CTASNKNRGIIKT

SEQ ID NO: 32 - protein, 15 aa, Artificial Sequence
  Respiratory Syncitial Virus
ASNKNRGIIKTFSNG

SEQ ID NO: 33 - protein, 13 aa, Artificial Sequence
  Respiratory Syncitial Virus
NKNRGIIKTFSNG

SEQ ID NO: 34 - protein, 11 aa, Artificial Sequence
  Respiratory Syncitial Virus
NRGIIKTFSNG

SEQ ID NO: 35 - protein, 11 aa, Artificial Sequence
  Respiratory Syncitial Virus
SNKNRGIIKTF

SEQ ID NO: 36 - protein, 16 aa, Artificial Sequence
  Respiratory Syncitial Virus
CTASNKNSGIIKTFSN

SEQ ID NO: 37 - protein, 11 aa, Artificial Sequence
  Respiratory Syncitial Virus
CTASNKNRGII

SEQ ID NO: 38 - protein, 15 aa, Artificial Sequence
  Respiratory Syncitial Virus
CTASNKNRGIIKTFS

SEQ ID NO: 39 - protein, 12 aa, Artificial Sequence
  Respiratory Syncitial Virus
TASNKNRGIIKT

SEQ ID NO: 40 - protein, 14 aa, Artificial Sequence
  Respiratory Syncitial Virus
TASNKNRGIIKTFS

SEQ ID NO: 41 - protein, 14 aa, Artificial Sequence
  Respiratory Syncitial Virus
TASNENRGIIKTFS

SEQ ID NO: 42 - protein, 13 aa, Artificial Sequence
  Respiratory Syncitial Virus
TASNKNRGIIKTF

SEQ ID NO: 43 - protein, 14 aa, Artificial Sequence
  Respiratory Syncitial Virus
TASNKNRGIITTFS

SEQ ID NO: 44 - protein, 14 aa, Artificial Sequence
  Respiratory Syncitial Virus
TASNKNEGIIKTFS

SEQ ID NO: 45 - protein, 14 aa, Artificial Sequence
  Respiratory Syncitial Virus
TASNKNRGIIETFS

SEQ ID NO: 46 - DNA, 1725 bp, Artificial Sequence
  Optimized Coding Sequence
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 47 - DNA, 1725 bp, Artificial Sequence; identical to SEQ ID NO: 46
  Optimized Coding Sequence

SEQ ID NO: 48 - protein, 98 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCA

SEQ ID NO: 49 - protein, 120 aa, Artificial Sequence
  CDR-grafted heavy chain
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSS

SEQ ID NO: 50 - protein, 101 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 51 - protein, 111 aa, Artificial Sequence
  CDR-grafted light chain
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIK

SEQ ID NO: 52 - protein, 15 aa, Artificial Sequence
  Light chain CDR1 variant
RASQSVDLFGRSYMH

SEQ ID NO: 53 - protein, 15 aa, Artificial Sequence
  Light chain CDR1 variant
RASQSVDLFGKSYMH

SEQ ID NO: 54 - protein, 15 aa, Artificial Sequence
  Light chain CDR1 variant
RASQSVDLFGQSYMH

SEQ ID NO: 55 - protein, 15 aa, Artificial Sequence
  Light chain CDR1 variant
RASQSVDLFGVSYMH

SEQ ID NO: 56 - protein, 15 aa, Artificial Sequence
  Light chain CDR1 variant
RASQSVDHFGKSYVH

SEQ ID NO: 57 - protein, 15 aa, Artificial Sequence
  Light chain CDR1 variant
RASQSVDVFGRSYMH

SEQ ID NO: 58 - protein, 15 aa, Artificial Sequence
  Light chain CDR1 variant
RASQSVDLFGTSYMH

SEQ ID NO: 59 - protein, 15 aa, Artificial Sequence
  Light chain CDR1 variant
RASQSVDYLGRSYMH

SEQ ID NO: 60 - protein, 15 aa, Artificial Sequence
  Light chain CDR1 variant
RASQSVDLFGSSYMH

SEQ ID NO: 61 - protein, 15 aa, Artificial Sequence
  Light chain CDR1 variant
RASQSVDYSGRSYMH

SEQ ID NO: 62 - protein, 15 aa, Artificial Sequence
  Light chain CDR1 variant
RASQSVDFFGKSYMH

SEQ ID NO: 63 - protein, 111 aa, Artificial Sequence
  Light chain with CDR1 variant
DIVLTQSPASLAVSLGQRATIFCRASQSVDLFGRSYMHWFQQKPGQPPKLLIYAASNPESGIPARFTGSGSGTDFTLNIHPVEEEDAATYYCQQIIEDPWTFGGGTKLEIK

SEQ ID NO: 64 - protein, 111 aa, Artificial Sequence
  Light chain with CDR1 variant
DIVLTQSPASLAVSLGQRATIFCRASQSVDLFGKSYMHWFQQKPGQPPKLLIYAASNPESGIPARFTGSGSGTDFTLNIHPVEEEDAATYYCQQIIEDPWTFGGGTKLEIK

SEQ ID NO: 65 - protein, 111 aa, Artificial Sequence
  Light chain with CDR1 variant
DIVLTQSPASLAVSLGQRATIFCRASQSVDLFGQSYMHWFQQKPGQPPKLLIYAASNPESGIPARFTGSGSGTDFTLNIHPVEEEDAATYYCQQIIEDPWTFGGGTKLEIK

SEQ ID NO: 66 - protein, 111 aa, Artificial Sequence
  Light chain with CDR1 variant
DIVLTQSPASLAVSLGQRATIFCRASQSVDLFGVSYMHWFQQKPGQPPKLLIYAASNPESGIPARFTGSGSGTDFTLNIHPVEEEDAATYYCQQIIEDPWTFGGGTKLEIK

SEQ ID NO: 67 - protein, 111 aa, Artificial Sequence
  Light chain with CDR1 variant
DIVLTQSPASLAVSLGQRATIFCRASQSVDHFGKSYVHWFQQKPGQPPKLLIYAASNPESGIPARFTGSGSGTDFTLNIHPVEEEDAATYYCQQIIEDPWTFGGGTKLEIK

SEQ ID NO: 68 - protein, 111 aa, Artificial Sequence
  Light chain with CDR1 variant
DIVLTQSPASLAVSLGQRATIFCRASQSVDVFGRSYMHWFQQKPGQPPKLLIYAASNPESGIPARFTGSGSGTDFTLNIHPVEEEDAATYYCQQIIEDPWTFGGGTKLEIK

SEQ ID NO: 69 - protein, 111 aa, Artificial Sequence
  Light chain with CDR1 variant
DIVLTQSPASLAVSLGQRATIFCRASQSVDLFGTSYMHWFQQKPGQPPKLLIYAASNPESGIPARFTGSGSGTDFTLNIHPVEEEDAATYYCQQIIEDPWTFGGGTKLEIK

SEQ ID NO: 70 - protein, 111 aa, Artificial Sequence
  Light chain with CDR1 variant
DIVLTQSPASLAVSLGQRATIFCRASQSVDYLGRSYMHWFQQKPGQPPKLLIYAASNPESGIPARFTGSGSGTDFTLNIHPVEEEDAATYYCQQIIEDPWTFGGGTKLEIK

SEQ ID NO: 71 - protein, 111 aa, Artificial Sequence
  Light chain with CDR1 variant
DIVLTQSPASLAVSLGQRATIFCRASQSVDLFGSSYMHWFQQKPGQPPKLLIYAASNPESGIPARFTGSGSGTDFTLNIHPVEEEDAATYYCQQIIEDPWTFGGGTKLEIK

SEQ ID NO: 72 - protein, 111 aa, Artificial Sequence
  Light chain with CDR1 variant
DIVLTQSPASLAVSLGQRATIFCRASQSVDYSGRSYMHWFQQKPGQPPKLLIYAASNPESGIPARFTGSGSGTDFTLNIHPVEEEDAATYYCQQIIEDPWTFGGGTKLEIK

SEQ ID NO: 73 - protein, 111 aa, Artificial Sequence
  Light chain with CDR1 variant
DIVLTQSPASLAVSLGQRATIFCRASQSVDFFGKSYMHWFQQKPGQPPKLLIYAASNPESGIPARFTGSGSGTDFTLNIHPVEEEDAATYYCQQIIEDPWTFGGGTKLEIK

SEQ ID NO: 74 - protein, 120 aa, Artificial Sequence
  Heavy chain with CDR2 variant
QITLKESGPTLVKPTQTLTLTCTFSGFSLSTSGMGVSWIRQPPGKALEWLAHLYWDDDKRYNPSLKSRLTITKDTSKNQVVLTMTNMDPVDTATYYCARLYGFTYGFAYWGQGTLVTVSS

SEQ ID NO: 75 - protein, 120 aa, Artificial Sequence
  Heavy chain with CDR3 variant
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLWGFRYGFAYWGQGTLVTVSS

SEQ ID NO: 76 - protein, 120 aa, Artificial Sequence
  Heavy chain with CDR3 variant
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLWGFKYGFAYWGQGTLVTVSS

SEQ ID NO: 77 - protein, 120 aa, Artificial Sequence
  Heavy chain with CDR3 variant
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLWGFAYGFAYWGQGTLVTVSS

SEQ ID NO: 78 - protein, 120 aa, Artificial Sequence
  Heavy chain with CDR3 variant
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFRYGFAYWGQGTLVTVSS

SEQ ID NO: 79 - protein, 120 aa, Artificial Sequence
  Heavy chain with CDR3 variant
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFKYGFAYWGQGTLVTVSS

SEQ ID NO: 80 - protein, 120 aa, Artificial Sequence
  Heavy chain with CDR3 variant
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFAYGFAYWGQGTLVTVSS

SEQ ID NO: 81 - protein, 111 aa, Artificial Sequence
  Light chain with CDR1 variant
DIVMTQSPDSLAVSLGERATINCRASQSVDLFGKSYMHWYQQKPGQPPKLLIYAASNPESGVPDRFSGSGSGTDFTLTISSLQAEDVAVYYCQQIIDDPWTFGQGTKVEIK

SEQ ID NO: 82 - protein, 111 aa, Artificial Sequence
  Light chain with CDR1 variant
DIVMTQSPDSLAVSLGERATINCRASDSVDLFGKSYMHWYQQKPGQPPKLLIYAASNPESGVPDRFSGSGSGTDFTLTISSLQAEDVAVYYCQQIIDDPWTFGQGTKVEIK

SEQ ID NO: 83 - protein, 111 aa, Artificial Sequence
  Light chain with CDR1 variant
DIVMTQSPDSLAVSLGERATINCRASDSVDLFGRSYMHWYQQKPGQPPKLLIYAASNPESGVPDRFSGSGSGTDFTLTISSLQAEDVAVYYCQQIIDDPWTFGQGTKVEIK

SEQ ID NO: 84 - protein, 120 aa, Artificial Sequence
  Heavy chain CDR2 variant
QITLKESGPTLVKPTQTLTLTCTFSGFSLSTSGMGVSWIRQPPGKALEWLAHLYWDDDYRYNPSLKSRLTITKDTSKNQVVLTMTNMDPVDTATYYCARLYGFTYGFAYWGQGTLVTVSS

SEQ ID NO: 85 - protein, 111 aa, Artificial Sequence
  Light chain with CDR1 and CDR3 variant
DIVMTQSPDSLAVSLGERATINCRASDSVDLFGQSYMHWYQQKPGQPPKLLIYAASNPESGVPDRFSGSGSGTDFTLTISSLQAEDVAVYYCQQIIDDPWTFGQGTKVEIK

SEQ ID NO: 86 - protein, 120 aa, Artificial Sequence; identical to SEQ ID NO: 74
  Heavy chain with CDR2 variant

SEQ ID NO: 87 - protein, 111 aa, Artificial Sequence
  Light chain with CDR1 and CDR3 variant
DIVMTQSPDSLAVSLGERATINCRASDSVDLFGVSYMHWYQQKPGQPPKLLIYAASNPESGVPDRFSGSGSGTDFTLTISSLQAEDVAVYYCQQIIDDPWTFGQGTKVEIK

SEQ ID NO: 88 - protein, 120 aa, Artificial Sequence
  Heavy chain with CDR2 variant
QITLKESGPTLVKPTQTLTLTCTFSGFSLSTSGMGVSWIRQPPGKALEWLAHIYWDDDKRYSPSLKSRLTITKDTSKNQVVLTMTNMDPVDTATYYCARLYGFTYGFAYWGQGTLVTVSS

SEQ ID NO: 89 - protein, 111 aa, Artificial Sequence
  Light chain with CDR1 and CDR3 variant
DIVMTQSPDSLAVSLGERATINCRASQSVDFFGTSYMHWYQQKPGQPPKLLIYAASNPESGVPDRFSGSGSGTDFTLTISSLQAEDVAVYYCQQIIDDPWTFGQGTKVEIK

SEQ ID NO: 90 - protein, 111 aa, Artificial Sequence
  Light chain with CDR1 variant
DIVMTQSPDSLAVSLGERATINCRASDSVDLFGISYQHWYQQKPGQPPKLLIYAASNPESGVPDRFSGSGSGTDFTLTISSLQAEDVAVYYCQQIIEDPWTFGQGTKVEIK

SEQ ID NO: 91 - protein, 111 aa, Artificial Sequence
  Light chain with CDR1 and CDR3 variant
DIVMTQSPDSLAVSLGERATINCRASQSVDLFGQSYMHWYQQKPGQPPKLLIYAASNPESGVPDRFSGSGSGTDFTLTISSLQAEDVAVYYCQQIIDDPWTFGQGTKVEIK

SEQ ID NO: 92 - protein, 111 aa, Artificial Sequence
  Light chain with CDR1 and CDR3 variant
DIVMTQSPDSLAVSLGERATINCRASHSVDLFGTSYMHWYQQKPGQPPKLLIYAASNPESGVPDRFSGSGSGTDFTLTISSLQAEDVAVYYCQQIIDDPWTFGQGTKVEIK

SEQ ID NO: 93 - protein, 120 aa, Artificial Sequence
  Heavy chain with CDR2 variant
QITLKESGPTLVKPTQTLTLTCTFSGFSLSTSGMGVSWIRQPPGKALEWLAHLYWDDDKRYSPSLKSRLTITKDTSKNQVVLTMTNMDPVDTATYYCARLYGFTYGFAYWGQGTLVTVSS

SEQ ID NO: 94 - protein, 111 aa, Artificial Sequence
  Light chain with CDR1 and CDR3 variant
DIVMTQSPDSLAVSLGERATINCRASDSVDLFGTSYMHWYQQKPGQPPKLLIYAASNPESGVPDRFSGSGSGTDFTLTISSLQAEDVAVYYCQQIIDDPWTFGQGTKVEIK

SEQ ID NO: 95 - protein, 111 aa, Artificial Sequence
  Light chain with CDR1 and CDR3 variant
DIVMTQSPDSLAVSLGERATINCRASQSVDLFGTSYMHWYQQKPGQPPKLLIYAASNPESGVPDRFSGSGSGTDFTLTISSLQAEDVAVYYCQQIIDDPWTFGQGTKVEIK

SEQ ID NO: 96 - protein, 111 aa, Artificial Sequence
  Light chain with CDR1 and CDR3 variant
DIVMTQSPDSLAVSLGERATINCRASQSVDFFGKSYMHWYQQKPGQPPKLLIYAASNPESGVPDRFSGSGSGTDFTLTISSLQAEDVAVYYCQQIIDDPWTFGQGTKVEIK

SEQ ID NO: 97 - protein, 111 aa, Artificial Sequence
  Light chain with CDR1 and CDR3 variant
DIVMTQSPDSLAVSLGERATINCRASDSVDFFGKSYMHWYQQKPGQPPKLLIYAASNPESGVPDRFSGSGSGTDFTLTISSLQAEDVAVYYCQQIIDDPWTFGQGTKVEIK

SEQ ID NO: 98 - protein, 111 aa, Artificial Sequence
  Light chain with CDR1 variant
DIVMTQSPDSLAVSLGERATINCRASDSVDLFGRSYMHWYQQKPGQPPKLLIYAASNPESGVPDRFSGSGSGTDFTLTISSLQAEDVAVYYCQQIIEDPWTFGQGTKVEIK

SEQ ID NO: 99 - protein, 111 aa, Artificial Sequence
  Light chain with CDR1 and CDR3 variant
DIVMTQSPDSLAVSLGERATINCRASQSVDYFGVSYMHWYQQKPGQPPKLLIYAASNPESGVPDRFSGSGSGTDFTLTISSLQAEDVAVYYCQQIIDDPWTFGQGTKVEIK

SEQ ID NO: 100 - protein, 111 aa, Artificial Sequence
  Light chain with CDR1 and CDR3 variant
DIVMTQSPDSLAVSLGERATINCRASDSVDYFGVSYMHWYQQKPGQPPKLLIYAASNPESGVPDRFSGSGSGTDFTLTISSLQAEDVAVYYCQQIIDDPWTFGQGTKVEIK

SEQ ID NO: 101 - protein, 111 aa, Artificial Sequence
  Light chain with CDR1 and CDR3 variant
DIVMTQSPDSLAVSLGERATINCRASQSVDYLGKSYMHWYQQKPGQPPKLLIYAASNPESGVPDRFSGSGSGTDFTLTISSLQAEDVAVYYCQQIIDDPWTFGQGTKVEIK

SEQ ID NO: 102 - protein, 110 aa, Artificial Sequence
  Light chain with CDR1 and CDR3 variant
DIVMTQSPDSLAVSLGERATINCRASQSVDYFGRSYMHWYQQKPGQPPKLLIYAASNPESGVPDRFSGSGSGTDFTLTISSLQAEDVAVYYCQQIIDDPWTFGQGTKVEI

SEQ ID NO: 103 - protein, 111 aa, Artificial Sequence
  Light chain with CDR1 and CDR3 variant
DIVMTQSPDSLAVSLGERATINCRASQSVDYFGTSYMHWYQQKPGQPPKLLIYAASNPESGVPDRFSGSGSGTDFTLTISSLQAEDVAVYYCQQIIDDPWTFGQGTKVEIK

SEQ ID NO: 104 - protein, 111 aa, Artificial Sequence
  Light chain with CDR1 variant
DIVMTQSPDSLAVSLGERATINCRASQSVDLLGGSYMHWYQQKPGQPPKLLIYAASNPESGVPDRFSGSGSGTDFTLTISSLQAEDVAVYYCQQIIEDPWTFGQGTKVEIK

SEQ ID NO: 105 - protein, 15 aa, Artificial Sequence
  Light chain CDR1 variant
RASQSVDLLGGSYMH

SEQ ID NO: 106 - protein, 111 aa, Artificial Sequence
  Light chain with CDR3 variant
DIVMTQSPDSLAVSLGERATINCRASQSVDYNGISYMHWYQQKPGQPPKLLIYAASNPESGVPDRFSGSGSGTDFTLTISSLQAEDVAVYYCQQIIDDPWTFGQGTKVEIK

SEQ ID NO: 107 - protein, 9 aa, Artificial Sequence
  Light chain CDR3 variant
QQIIDDPWT

SEQ ID NO: 108 - protein, 111 aa, Artificial Sequence
  Light chain with CDR1 and CDR3 variant
DIVMTQSPDSLAVSLGERATINCRASDSVDYFGQSYMHWYQQKPGQPPKLLIYAASNPESGVPDRFSGSGSGTDFTLTISSLQAEDVAVYYCQQIIDDPWTFGQGTKVEIK

SEQ ID NO: 109 - protein, 111 aa, Artificial Sequence
  Light chain with CDR1 and CDR3 variant
DIVMTQSPDSLAVSLGERATINCRASDSVDLFGQSYMHWYQQKPGQPPKLLIYAASNPESGVPDRFSGSGSGTDFTLTISSLQAEDVAVYYCQQIIADPWTFGQGTKVEIK

SEQ ID NO: 110 - protein, 9 aa, Artificial Sequence
  Light chain CDR3 variant
QQIIADPWT

SEQ ID NO: 111 - protein, 111 aa, Artificial Sequence
  Light chain with CDR1 variant
DIVMTQSPDSLAVSLGERATINCRASDSVDLFGVSYMHWYQQKPGQPPKLLIYAASNPESGVPDRFSGSGSGTDFTLTISSLQAEDVAVYYCQQIIEDPWTFGQGTKVEIK

SEQ ID NO: 112 - protein, 111 aa, Artificial Sequence
  Light chain with CDR1 and CDR3 variant
DIVMTQSPDSLAVSLGERATINCRASDSVDYFGRSYMHWYQQKPGQPPKLLIYAASNPESGVPDRFSGSGSGTDFTLTISSLQAEDVAVYYCQQIIDDPWTFGQGTKVEIK